SEQUENCE LISTING (180 sequences)

SEQ ID NO: 1 - protein, 122 aa, synthetic construct
EVQLLESGGGLVQPGGSLRLSCAASGFTFSTFAMSWVRQAPGKGLEWVSTISGGGDSTYYADSVKGRFTISRDNSKNMMSLQMNSLRAVDTAVYYCVKGGGDEYFYYGMDVWGQGTTVTVSS

SEQ ID NO: 2 - protein, 126 aa, synthetic construct
QVQLVQSGAEVKKPGASVKVSCKASGYTFTSYDINWVRQATGQGLEWMGWMNPNSGNTGYAQNFQGRVTMTRNTSINTAYMELSSLRSEDTAVYYCTRGGPTLVRGVIIRDAFDFWGQGTMVTVSS

SEQ ID NO: 3 - protein, 123 aa, synthetic construct
QVQLVQSGTEVKKPGASVKVSCKVSGYTLSDLSIHWVRQAPGKGLEWMGGFEPEDGEIIYAQKFQGRVIMTEDTSTDTAYMKLSSLRSEDTAVYYCATDKRYFDWLLSGFDYWGQGTLVTVSS

SEQ ID NO: 4 - protein, 117 aa, synthetic construct
QGQLVQSGAEVKKPGASVKVSCKASGYTFNNYGISWVRQAPGQGLEWMGWISAYNGFTRSTQKFQGRVTLTTDTSTSTAHMELRSLRSDDTAAYYCARDGGDGFDPWGQGTLVTVSS

SEQ ID NO: 5 - protein, 120 aa, synthetic construct
QVQLVESGGGVVQPGRSLRLSCAASGFTFSSYGMHWVRQAPGKGLEWVAVISYDGSNKYYTDSVKGRFTISRDNSKNTLYLQMNSLRAEDTAVYYCARGGYYGSGSLDYWGQGTLVTVSS

SEQ ID NO: 6 - protein, 126 aa, synthetic construct
QVQLVQSGAEVKKPGASVKVSCKASGYTFTSYDINWVRQATGQGLEWMGWMNPNSGNTGYAQNFQGRVTMTRDTSVNTAYMELSSLRSEDTAVYYCARGGPNLVRGVIIRDAFDFWGQGTMVTVSS

SEQ ID NO: 7 - protein, 118 aa, synthetic construct
  MOD_RES 89: note = Any amino acid
QVQLVESGGGVVQPGRSLRLSCAASGFTFSIYGMHWVRQAPGKGLEWVAVISNDGSNEYYADSVKGRFTISRDYSKNTLYLQMNSLRAXDTAVYYCAREGESYPFDYWGQGTLVTVSS

SEQ ID NO: 8 - protein, 109 aa, synthetic construct
DIQMTQSPSSLSASVGDRVTITCRASQGIRNDLGWYQQKPGKAPKRLIYSASSLQSGVPSRFSGSGSGTEFTLTISSLQPEDFATYYCLQHNSYPYTFGQGTPLDIKRA

SEQ ID NO: 9 - protein, 109 aa, synthetic construct
EIVMTQSPATLSVSPGERATLSCRASQSVSNSFAWYQQKPGQAPRLLIYGAFTRATGIPARFSGSGSGTEFTLTISSLQSEDFAVYYCQQYKTWPLTFGGGTKVEIKRA

SEQ ID NO: 10 - protein, 109 aa, synthetic construct
DIQLTQSPSFLSASVGDRVTITCRASQGISSYLAWYQQKPGKAPKLLIFAASTLQSGVPSRFSGSGSGTEFTLTISSLQPEDFATYYCQRLHSYPRTFGQGTKVEIKRA

SEQ ID NO: 11 - protein, 108 aa, synthetic construct
EIVLTQSPATLSLSPGERATLSCRASQSVSSFLAWYQQKPGQAPRLLIFDASNRATGIPARFSGSGSGTDFTLTISSLEPEDFAVYYCQQRSNWITFGQGTRLEIKRA

SEQ ID NO: 12 - protein, 114 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIKRA

SEQ ID NO: 13 - protein, 109 aa, synthetic construct
EIVMTQSPATLSVSPGERATLSCRASQSVSNSFAWYQQKPGQAPRLLIYGAFTRATGIPARFSGSGSGIEFTLTISSLQSEDFAVYYCQQYKTWPLTFGGGTKVEIKRA

SEQ ID NO: 14 - protein, 109 aa, synthetic construct
EIVMTQSPATLSVSPGERATLSCRASQSVSSNLAWYQQKPGQAPRLLIYGAFFRATGIPARFSGSGSGTEFTLTISSLQSEDFAVYYCQQYNNWPYTFGQGTKLEIKRA

SEQ ID NO: 15 - DNA, 366 bp, synthetic construct
  source 1..366: mol_type = other DNA
gaggtgcagctgttggagtctgggggaggcttggtacagccgggggggtccctgagactctcctgtgcagcctctggattcacctttagtacctttgccatgagctgggtccgccaggctccaggaaaggggctggagtgggtctcaactattagtggtggtggtgacagtacatactacgcagactccgtgaagggccggttcaccatctccagagacaattccaagaacatgatgtctctacaaatgaacagcctgagagccgtggacacggccgtatattactgtgtgaagggggggggagacgaatacttctactacggtatggacgtctggggccaagggaccacggtcaccgtctcctca

SEQ ID NO: 16 - DNA, 378 bp, synthetic construct
  source 1..378: mol_type = other DNA
caggtacagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaatttccagggcagagtcaccatgaccaggaacacctccattaacacagcctacatggagctgagcagcctgagatctgaggacacggccgtgtattactgtacgagaggggggcctactctggttcggggagttattataagagatgcttttgatttctggggccaagggacaatggtcaccgtctcctca

SEQ ID NO: 17 - DNA, 369 bp, synthetic construct
  source 1..369: mol_type = other DNA
caggtccagttggtccagtctgggactgaggtgaagaagccgggggcctcagtgaaggtctcctgcaaagtttccggatacaccctcagtgacttatccattcactgggtgcgacaggctcctggaaaagggcttgagtggatgggaggttttgaacctgaagatggtgaaataatctacgcacagaagttccagggcagagtcattatgaccgaggacacatctacagacacagcctacatgaaactgagcagcctgagatctgaggacacggccgtgtattattgtgcaacagataaacgatattttgactggttattaagcggctttgactattggggccagggaaccctggtcaccgtctcctca

SEQ ID NO: 18 - DNA, 351 bp, synthetic construct
  source 1..351: mol_type = other DNA
cagggtcagctggtgcagtctggagctgaggtgaagaagcctggggcctcagtgaaggtctcctgcaaggcttctggttacacctttaacaactatggtatcagttgggtgcgacaggcccctggacaagggcttgagtggatgggatggatcagcgcttataatggtttcacaagaagtacacagaaattccagggcagagtcaccttgaccacagacacatcaacgagcacagcccacatggagttgaggagcctgagatctgacgacacggccgcttattattgtgcgagagatgggggagacgggttcgacccctggggccagggaaccctggtcaccgtctcctca

SEQ ID NO: 19 - DNA, 360 bp, synthetic construct
  source 1..360: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatacagactccgtgaagggccgattcaccatctccagagacaattccaagaacacgctgtatctgcaaatgaacagcctgagagctgaggacacggctgtgtattactgtgcgagagggggttactatggttcggggagccttgactactggggccagggaaccctggtcaccgtctcctca

SEQ ID NO: 20 - DNA, 378 bp, synthetic construct
  source 1..378: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaatttccagggcagagtcaccatgaccagggacacctccgtaaacacagcctacatggagctgagcagcctgagatctgaggacacggccgtgtattactgtgcgagaggggggcctaatctggttcggggagttattataagagatgcttttgatttctggggccaagggacaatggtcaccgtctcctca

SEQ ID NO: 21 - DNA, 354 bp, synthetic construct
  source 1..354: mol_type = other DNA
  variation 267: note = a, c, t, g, unknown or other
caggtgcaactggtggagtctgggggaggcgtggtccagcctgggaggtccctgagactctcctgtgcagcctctggattcaccttcagtatctatggcatgcactgggtccgccaggctccaggcaaggggctggagtgggtggcagttatatcaaatgatggaagtaatgaatactatgcagactccgtgaagggccgattcaccatctccagagactattccaagaacacgctgtatctgcaaatgaacagcctgagagctgangacacggctgtgtattattgtgcgagagagggggagagctacccctttgactactggggccagggaaccctggtcaccgtctcctca

SEQ ID NO: 22 - DNA, 327 bp, synthetic construct
  source 1..327: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctattctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccccgctggacatcaaacgggct

SEQ ID NO: 23 - DNA, 327 bp, synthetic construct
  source 1..327: mol_type = other DNA
gaaatagtgatgacgcagtctccagccaccctgtctgtgtctccaggggaaagagccaccctctcctgcagggccagtcagagtgttagcaacagctttgcctggtaccagcagaaacctggccaggctcccaggctcctcatctatggtgcattcaccagggccactggtatcccagccaggttcagtggcagtgggtctgggacagagttcactctcaccatcagcagcctgcagtctgaagattttgcagtttattactgtcagcagtataagacctggcctctcactttcggcggagggaccaaggtggagatcaaacgggct

SEQ ID NO: 24 - DNA, 327 bp, synthetic construct
  source 1..327: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctttgctgcatccactttgcaaagtggggtcccatcaaggttcagcggcagtggatctgggacagaattcactctcacaatcagcagcctgcagcctgaggattttgcaacttattactgtcaacgacttcatagttaccctcggacgttcggccaagggaccaaggtggaaatcaaacgggct

SEQ ID NO: 25 - DNA, 325 bp, synthetic construct
  source 1..325: mol_type = other DNA
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctttgatgcatccaacagggccactggcatcccagccaggttcagtggcagtgggtctgggacagacttcactctcaccatcagcagcctagagcctgaagattttgcagtttattactgtcagcagcgtagcaactggatcaccttcggccaagggacacgactggagattaaacgggctg

SEQ ID NO: 26 - DNA, 342 bp, synthetic construct
  source 1..342: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaacgggct

SEQ ID NO: 27 - DNA, 327 bp, synthetic construct
  source 1..327: mol_type = other DNA
gaaatagtgatgacgcagtctccagccaccctgtctgtgtctccaggggaaagagccaccctctcctgcagggccagtcagagtgttagcaacagctttgcctggtaccagcagaaacctggccaggctcccaggctcctcatctatggtgcattcaccagggccactggtatcccagccaggttcagtggcagtgggtctgggatagagttcactctcaccatcagcagcctgcagtctgaagattttgcagtttattactgtcagcagtataagacctggcctctcactttcggcggagggaccaaggtggagatcaaacgggct

SEQ ID NO: 28 - DNA, 327 bp, synthetic construct
  source 1..327: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcattttttagggccactggtatcccagccaggttcagtggcagtgggtctgggacagagttcactctcaccatcagcagcctgcagtctgaagattttgcagtttattactgtcagcagtataataactggccgtacacttttggccaggggaccaagctggagatcaaacgggct

SEQ ID NO: 29 - protein, 5 aa, synthetic construct
TFAMS

SEQ ID NO: 30 - protein, 5 aa, synthetic construct
SYDIN

SEQ ID NO: 31 - protein, 5 aa, synthetic construct
DLSIH

SEQ ID NO: 32 - protein, 5 aa, synthetic construct
NYGIS

SEQ ID NO: 33 - protein, 5 aa, synthetic construct
SYGMH

SEQ ID NO: 34 - protein, 5 aa, synthetic construct
IYGMH

SEQ ID NO: 35 - protein, 17 aa, synthetic construct
TISGGGDSTYYADSVKG

SEQ ID NO: 36 - protein, 17 aa, synthetic construct
WMNPNSGNTGYAQNFQG

SEQ ID NO: 37 - protein, 17 aa, synthetic construct
GFEPEDGEIIYAQKFQG

SEQ ID NO: 38 - protein, 17 aa, synthetic construct
WISAYNGFTRSTQKFQG

SEQ ID NO: 39 - protein, 17 aa, synthetic construct
VISYDGSNKYYTDSVKG

SEQ ID NO: 40 - protein, 17 aa, synthetic construct
VISNDGSNEYYADSVKG

SEQ ID NO: 41 - protein, 13 aa, synthetic construct
GGGDEYFYYGMDV

SEQ ID NO: 42 - protein, 17 aa, synthetic construct
GGPTLVRGVIIRDAFDF

SEQ ID NO: 43 - protein, 14 aa, synthetic construct
DKRYFDWLLSGFDY

SEQ ID NO: 44 - protein, 8 aa, synthetic construct
DGGDGFDP

SEQ ID NO: 45 - protein, 11 aa, synthetic construct
GGYYGSGSLDY

SEQ ID NO: 46 - protein, 17 aa, synthetic construct
GGPNLVRGVIIRDAFDF

SEQ ID NO: 47 - protein, 9 aa, synthetic construct
EGESYPFDY

SEQ ID NO: 48 - protein, 11 aa, synthetic construct
RASQGIRNDLG

SEQ ID NO: 49 - protein, 11 aa, synthetic construct
RASQSVSNSFA

SEQ ID NO: 50 - protein, 11 aa, synthetic construct
RASQGISSYLA

SEQ ID NO: 51 - protein, 11 aa, synthetic construct
RASQSVSSFLA

SEQ ID NO: 52 - protein, 16 aa, synthetic construct
RSSQSLVYSDGNTYLN

SEQ ID NO: 53 - protein, 11 aa, synthetic construct
RASQSVSSNLA

SEQ ID NO: 54 - protein, 7 aa, synthetic construct
SASSLQS

SEQ ID NO: 55 - protein, 7 aa, synthetic construct
GAFTRAT

SEQ ID NO: 56 - protein, 7 aa, synthetic construct
AASTLQS

SEQ ID NO: 57 - protein, 7 aa, synthetic construct
DASNRAT

SEQ ID NO: 58 - protein, 7 aa, synthetic construct
KVSNRDS

SEQ ID NO: 59 - protein, 7 aa, synthetic construct
GAFFRAT

SEQ ID NO: 60 - protein, 9 aa, synthetic construct
LQHNSYPYT

SEQ ID NO: 61 - protein, 9 aa, synthetic construct
QQYKTWPLT

SEQ ID NO: 62 - protein, 9 aa, synthetic construct
QRLHSYPRT

SEQ ID NO: 63 - protein, 8 aa, synthetic construct
QQRSNWIT

SEQ ID NO: 64 - protein, 9 aa, synthetic construct
MQGTHWPWT

SEQ ID NO: 65 - protein, 9 aa, synthetic construct
QQYNNWPYT

SEQ ID NO: 66 - protein, 8 aa, synthetic construct
GFTFSTFA

SEQ ID NO: 67 - protein, 8 aa, synthetic construct
GYTFTSYD

SEQ ID NO: 68 - protein, 8 aa, synthetic construct
GYTLSDLS

SEQ ID NO: 69 - protein, 8 aa, synthetic construct
GYTFNNYG

SEQ ID NO: 70 - protein, 8 aa, synthetic construct
GFTFSSYG

SEQ ID NO: 71 - protein, 8 aa, synthetic construct
GFTFSIYG

SEQ ID NO: 72 - protein, 8 aa, synthetic construct
ISGGGDST

SEQ ID NO: 73 - protein, 8 aa, synthetic construct
MNPNSGNT

SEQ ID NO: 74 - protein, 8 aa, synthetic construct
FEPEDGEI

SEQ ID NO: 75 - protein, 8 aa, synthetic construct
ISAYNGFT

SEQ ID NO: 76 - protein, 8 aa, synthetic construct
ISYDGSNK

SEQ ID NO: 77 - protein, 8 aa, synthetic construct
ISNDGSNE

SEQ ID NO: 78 - protein, 15 aa, synthetic construct
VKGGGDEYFYYGMDV

SEQ ID NO: 79 - protein, 19 aa, synthetic construct
TRGGPTLVRGVIIRDAFDF

SEQ ID NO: 80 - protein, 16 aa, synthetic construct
ATDKRYFDWLLSGFDY

SEQ ID NO: 81 - protein, 10 aa, synthetic construct
ARDGGDGFDP

SEQ ID NO: 82 - protein, 13 aa, synthetic construct
ARGGYYGSGSLDY

SEQ ID NO: 83 - protein, 19 aa, synthetic construct
ARGGPNLVRGVIIRDAFDF

SEQ ID NO: 84 - protein, 11 aa, synthetic construct
AREGESYPFDY

SEQ ID NO: 85 - protein, 6 aa, synthetic construct
QGIRND

SEQ ID NO: 86 - protein, 6 aa, synthetic construct
QSVSNS

SEQ ID NO: 87 - protein, 6 aa, synthetic construct
QGISSY

SEQ ID NO: 88 - protein, 6 aa, synthetic construct
QSVSSF

SEQ ID NO: 89 - protein, 11 aa, synthetic construct
QSLVYSDGNTY

SEQ ID NO: 90 - protein, 6 aa, synthetic construct
QSVSSN

SEQ ID NO: 91-95 - skipped (5 entries)

SEQ ID NO: 96 - protein, 30 aa, synthetic construct
EVQLLESGGGLVQPGGSLRLSCAASGFTFS

SEQ ID NO: 97 - protein, 30 aa, synthetic construct
QVQLVQSGAEVKKPGASVKVSCKASGYTFT

SEQ ID NO: 98 - protein, 30 aa, synthetic construct
QVQLVQSGTEVKKPGASVKVSCKVSGYTLS

SEQ ID NO: 99 - protein, 30 aa, synthetic construct
QGQLVQSGAEVKKPGASVKVSCKASGYTFN

SEQ ID NO: 100 - protein, 30 aa, synthetic construct
QVQLVESGGGVVQPGRSLRLSCAASGFTFS

SEQ ID NO: 101 - protein, 14 aa, synthetic construct
WVRQAPGKGLEWVS

SEQ ID NO: 102 - protein, 14 aa, synthetic construct
WVRQATGQGLEWMG

SEQ ID NO: 103 - protein, 14 aa, synthetic construct
WVRQAPGKGLEWMG

SEQ ID NO: 104 - protein, 14 aa, synthetic construct
WVRQAPGQGLEWMG

SEQ ID NO: 105 - protein, 14 aa, synthetic construct
WVRQAPGKGLEWVA

SEQ ID NO: 106 - protein, 32 aa, synthetic construct
RFTISRDNSKNMMSLQMNSLRAVDTAVYYCVK

SEQ ID NO: 107 - protein, 32 aa, synthetic construct
RVTMTRNTSINTAYMELSSLRSEDTAVYYCTR

SEQ ID NO: 108 - protein, 32 aa, synthetic construct
RVIMTEDTSTDTAYMKLSSLRSEDTAVYYCAT

SEQ ID NO: 109 - protein, 32 aa, synthetic construct
RVTLTTDTSTSTAHMELRSLRSDDTAAYYCAR

SEQ ID NO: 110 - protein, 32 aa, synthetic construct
RFTISRDNSKNTLYLQMNSLRAEDTAVYYCAR

SEQ ID NO: 111 - protein, 32 aa, synthetic construct
RVTMTRDTSVNTAYMELSSLRSEDTAVYYCAR

SEQ ID NO: 112 - protein, 32 aa, synthetic construct
  VARIANT 23: note = Any amino acid
RFTISRDYSKNTLYLQMNSLRAXDTAVYYCAR

SEQ ID NO: 113 - protein, 11 aa, synthetic construct
WGQGTTVTVSS

SEQ ID NO: 114 - protein, 11 aa, synthetic construct
WGQGTMVTVSS

SEQ ID NO: 115 - protein, 11 aa, synthetic construct
WGQGTLVTVSS

SEQ ID NO: 116 - protein, 23 aa, synthetic construct
DIQMTQSPSSLSASVGDRVTITC

SEQ ID NO: 117 - protein, 23 aa, synthetic construct
EIVMTQSPATLSVSPGERATLSC

SEQ ID NO: 118 - protein, 23 aa, synthetic construct
DIQLTQSPSFLSASVGDRVTITC

SEQ ID NO: 119 - protein, 23 aa, synthetic construct
EIVLTQSPATLSLSPGERATLSC

SEQ ID NO: 120 - protein, 23 aa, synthetic construct
DVVMTQSPLSLPVTLGQPASISC

SEQ ID NO: 121 - protein, 15 aa, synthetic construct
WYQQKPGKAPKRLIY

SEQ ID NO: 122 - protein, 15 aa, synthetic construct
WYQQKPGQAPRLLIY

SEQ ID NO: 123 - protein, 15 aa, synthetic construct
WYQQKPGKAPKLLIF

SEQ ID NO: 124 - protein, 15 aa, synthetic construct
WYQQKPGQAPRLLIF

SEQ ID NO: 125 - protein, 15 aa, synthetic construct
WFQQRPGQSPRRLIY

SEQ ID NO: 126 - protein, 32 aa, synthetic construct
GVPSRFSGSGSGTEFTLTISSLQPEDFATYYC

SEQ ID NO: 127 - protein, 32 aa, synthetic construct
GIPARFSGSGSGTEFTLTISSLQSEDFAVYYC

SEQ ID NO: 128 - protein, 32 aa, synthetic construct
GIPARFSGSGSGTDFTLTISSLEPEDFAVYYC

SEQ ID NO: 129 - protein, 32 aa, synthetic construct
GVPDRFSGSGSGTDFTLKISRVEAEDVGVYYC

SEQ ID NO: 130 - protein, 32 aa, synthetic construct
GIPARFSGSGSGIEFTLTISSLQSEDFAVYYC

SEQ ID NO: 131 - protein, 12 aa, synthetic construct
FGQGTPLDIKRA

SEQ ID NO: 132 - protein, 12 aa, synthetic construct
FGGGTKVEIKRA

SEQ ID NO: 133 - protein, 12 aa, synthetic construct
FGQGTKVEIKRA

SEQ ID NO: 134 - protein, 12 aa, synthetic construct
FGQGTRLEIKRA

SEQ ID NO: 135 - protein, 12 aa, synthetic construct
FGQGTKLEIKRA

SEQ ID NO: 136 - protein, 25 aa, synthetic construct
EVQLLESGGGLVQPGGSLRLSCAAS

SEQ ID NO: 137 - protein, 25 aa, synthetic construct [sequence omitted]

SEQ ID NO: 138 - protein, 25 aa, synthetic construct
QVQLVQSGTEVKKPGASVKVSCKVS

SEQ ID NO: 139 - protein, 25 aa, synthetic construct
QGQLVQSGAEVKKPGASVKVSCKAS

SEQ ID NO: 140 - protein, 25 aa, synthetic construct
QVQLVESGGGVVQPGRSLRLSCAAS

SEQ ID NO: 141 - protein, 17 aa, synthetic construct
MSWVRQAPGKGLEWVST

SEQ ID NO: 142 - protein, 17 aa, synthetic construct
INWVRQATGQGLEWMGW

SEQ ID NO: 143 - protein, 17 aa, synthetic construct
IHWVRQAPGKGLEWMGG

SEQ ID NO: 144 - protein, 17 aa, synthetic construct
ISWVRQAPGQGLEWMGW

SEQ ID NO: 145 - protein, 17 aa, synthetic construct
MHWVRQAPGKGLEWVAV

SEQ ID NO: 146 - protein, 38 aa, synthetic construct
YYADSVKGRFTISRDNSKNMMSLQMNSLRAVDTAVYYC

SEQ ID NO: 147 - protein, 38 aa, synthetic construct
GYAQNFQGRVTMTRNTSINTAYMELSSLRSEDTAVYYC

SEQ ID NO: 148 - protein, 38 aa, synthetic construct
IYAQKFQGRVIMTEDTSTDTAYMKLSSLRSEDTAVYYC

SEQ ID NO: 149 - protein, 38 aa, synthetic construct
RSTQKFQGRVTLTTDTSTSTAHMELRSLRSDDTAAYYC

SEQ ID NO: 150 - protein, 38 aa, synthetic construct
YYTDSVKGRFTISRDNSKNTLYLQMNSLRAEDTAVYYC

SEQ ID NO: 151 - protein, 38 aa, synthetic construct
GYAQNFQGRVTMTRDTSVNTAYMELSSLRSEDTAVYYC

SEQ ID NO: 152 - protein, 38 aa, synthetic construct
  VARIANT 31: note = Any amino acid
YYADSVKGRFTISRDYSKNTLYLQMNSLRAXDTAVYYC

SEQ ID NO: 153 - protein, 26 aa, synthetic construct [sequence omitted]

SEQ ID NO: 154 - protein, 26 aa, synthetic construct
EIVMTQSPATLSVSPGERATLSCRAS

SEQ ID NO: 155 - protein, 26 aa, synthetic construct
DIQLTQSPSFLSASVGDRVTITCRAS

SEQ ID NO: 156 - protein, 26 aa, synthetic construct [sequence omitted]

SEQ ID NO: 157 - protein, 26 aa, synthetic construct
DVVMTQSPLSLPVTLGQPASISCRSS

SEQ ID NO: 158 - protein, 17 aa, synthetic construct
LGWYQQKPGKAPKRLIY

SEQ ID NO: 159 - protein, 17 aa, synthetic construct
FAWYQQKPGQAPRLLIY

SEQ ID NO: 160 - protein, 17 aa, synthetic construct
LAWYQQKPGKAPKLLIF

SEQ ID NO: 161 - protein, 17 aa, synthetic construct
LAWYQQKPGQAPRLLIF

SEQ ID NO: 162 - protein, 17 aa, synthetic construct
LNWFQQRPGQSPRRLIY

SEQ ID NO: 163 - protein, 17 aa, synthetic construct
LAWYQQKPGQAPRLLIY

SEQ ID NO: 164 - protein, 37 aa, synthetic construct
SSLQSGVPSRFSGSGSGTEFTLTISSLQPEDFATYYC

SEQ ID NO: 165 - protein, 37 aa, synthetic construct
FTRATGIPARFSGSGSGTEFTLTISSLQSEDFAVYYC

SEQ ID NO: 166 - protein, 37 aa, synthetic construct
STLQSGVPSRFSGSGSGTEFTLTISSLQPEDFATYYC

SEQ ID NO: 167 - protein, 37 aa, synthetic construct
SNRATGIPARFSGSGSGTDFTLTISSLEPEDFAVYYC

SEQ ID NO: 168 - protein, 37 aa, synthetic construct
SNRDSGVPDRFSGSGSGTDFTLKISRVEAEDVGVYYC

SEQ ID NO: 169 - protein, 37 aa, synthetic construct
FTRATGIPARFSGSGSGIEFTLTISSLQSEDFAVYYC

SEQ ID NO: 170 - protein, 37 aa, synthetic construct
FFRATGIPARFSGSGSGTEFTLTISSLQSEDFAVYYC

SEQ ID NO: 171 - protein, 481 aa, Homo sapiens
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGA

SEQ ID NO: 172 - protein, 595 aa, Homo sapiens
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 173 - protein, 477 aa, Homo sapiens
MLLPLLWANEERDSGGWADPRFSTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 174 - protein, 142 aa, Homo sapiens
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVT

SEQ ID NO: 175 - protein, 236 aa, synthetic construct
MLLLLLLLPPLLCGRVGAKEASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVR

SEQ ID NO: 176 - protein, 137 aa, synthetic construct
MLLLLLLLPPLLCGRVGAKEPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGA

SEQ ID NO: 177 - protein, 232 aa, Mus musculus
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 178 - protein, 238 aa, Mus musculus
EPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 179 - protein, 6 aa, synthetic construct
  source 1..6: note = 6xHis tag
HHHHHH

SEQ ID NO: 180 - protein, 8 aa, synthetic construct
WSHPQFEK

(19) United States
(12) Patent Application Publication
(10) Pub. No.: US 2025/0019432 A1
(43) Pub. Date: Jan. 16, 2025

(54) CELL-BINDING PROTEINS AND METHODS OF USE

(71) Applicant: MABLYTICS, INC., New York, NY (US)

(72) Inventors: Anthony Clark Stevens, San Diego, CA (US); Eric Richard SJOBERG, San Diego, CA (US)

(21) Appl. No.: 18/384,623

(22) Filed: Oct. 27, 2023

Related U.S. Application Data
(63) Continuation of application No. 18/089,631, filed on Dec. 28, 2022, now abandoned.
Application No. 18/089,631 is a continuation of application No. PCT/US21/40043, filed on Jul. 1, 2021.
(60) Provisional application No. 63/047,613, filed on Jul. 2, 2020.
(60) Provisional application No. 63/140,309, filed on Jan. 22, 2021.

(51) Int. Cl. C07K 16/28

(52) U.S. Cl. CPC C07K 16/2803; C07K 2317/24; C07K 2317/55; C07K 2317/565; C07K 2317/567; C07K 2317/92

(57) ABSTRACT

Embodiments of the present disclosure provide antibodies binding to sialic acid binding-immunoglobulin type lectin (siglec) proteins. Also provided are methods of treating and diagnosing cancer using such antibodies.

### CROSS REFERENCE TO RELATED APPLICATIONS

This application is a continuation of U.S. application Ser. No. 18/089,631, filed Dec. 28, 2022, which is a continuation of International Application No. PCT/US2021/040043, which designated the United States and was filed on Jul. 1, 2021, published in English, which claims priority to U.S. Provisional Application No. 63/047,613 filed on Jul. 2, 2020 entitled CELL-BINDING PROTEINS AND METHODS OF USE and U.S. Provisional Application No. 63/140,309 filed on Jan. 22, 2021 entitled CELL-BINDING PROTEINS AND METHODS OF USE, the contents of each of which are herein incorporated by reference in their entirety.

### SEQUENCE LISTING

The sequence listing submitted via EFS, in compliance with 37 CFR § 1.52(e)(5), is incorporated herein by reference. The sequence listing XML file submitted via EFS contains the file 43451000 US3 sub seq list.xml, created on Oct. 3, 2024, which is 165,315 bytes in size.

### BACKGROUND

The sialic acid binding-immunoglobulin type lectins (siglecs) are a large family of proteins consisting of fifteen members. They bind sialic acid glycans found on N-linked glycans and glycolipids (Duan and Paulson., 2020, Ann Rev Immunology 14, 365-395). Sialic acids are nine carbon anionic sugars with a C1 carboxyl group, a C5 N-acetyl group, and a glycerol side chain from C7-C9. Siglec extracellular domains include immunoglobulin (Ig) domains that are structurally similar to variable and constant regions of an antibody. CD33-related siglecs are family members thought to be derived from CD33 duplication of the CD33 gene. Sialic acid binding sites are present in the extracellular domains with a majority of siglecs having immunoreceptor tyrosine inhibitor motifs (ITIMs) within their intracellular domains. ITIMs are phosphorylated by src kinases and recruit src-homology 2 domain (SH2) containing phosphatases SHP-1 and SHP-2. SHP-1 and SHP-2 dephosphorylate signaling molecules in a variety of cellular activation signaling pathways.

Cancer is a leading cause of death worldwide. It is characterized by unchecked cell growth and proliferation. SHP2 is a known proto-oncogene shown to promote cell invasion, proliferation, and survival associated with various cancers. There remains a need in the field for compounds and methods to treat cancer, including SHP2-related cancers linked to abnormal siglec expression and/or function. The present disclosure addresses this need with related compounds and methods described herein.

### SUMMARY

In some embodiments, the present disclosure provides an antibody, wherein the antibody binds a sialic acid binding-immunoglobulin type lectin (siglec) and wherein the antibody includes at least one human antibody variable domain. The siglec may include siglec12. The antibody may include at least one complementarity determining region (CDR) amino acid sequence selected from Table 5, at least one variable domain comprising a CDR amino acid sequence set selected from Table 6, and/or a pair of variable domains comprising a CDR amino acid sequence set pair selected from Table 7. The antibody may include at least one antibody framework (FR) amino acid sequence selected from Table 8. The antibody may include a variable domain amino acid sequence selected from Table 2 or encoded by a nucleic acid sequence selected from Table 3. The antibody may include a variable domain amino acid sequence pair selected from Table 4. The antibody may bind to the siglec12 extracellular domain. The antibody may bind to the siglec12 long isoform extracellular domain, the siglec12 short isoform extracellular domain, a subdomain of the siglec12 long isoform extracellular domain, and/or a subdomain of the siglec12 short isoform extracellular domain. The subdomain of the siglec12 long isoform extracellular domain and/or the subdomain of the siglec12 short isoform extracellular domain may include the Vset1 (S12-V1) region, the Vset2 (S12-V2) region, the C2set1 (S12-C1) region, or the C2set2 (S12-C2) region. The antibody may bind to a combination of subdomains of the siglec12 extracellular domain. The combination of subdomains may include S12-V2 and S12-C1 (S12-V2C1) or S12-V2, S12-C1, and S12-C2 (S12-V2C1C2). The antibody may not bind to S12-V1 or S12-C2. The antibody may not bind to siglec7 or siglec9. The antibody may not bind to any siglec proteins other than siglec12. The siglec may be associated with a cell. The cell may be a cancer cell. The cancer cell may be from a cancer selected from any of those listed in Table 10. The cancer cell may be a leukemia cell. The leukemia cell may be an acute myeloid leukemia (AML) cell or a chronic myeloid leukemia (CML) cell. Antibody binding to cell-associated siglec may induce cell death. Antibody binding to cell-associate siglec may inhibit tumor immunosuppression. Antibody binding to cell-associated siglec may induce antibody-dependent cellular cytotoxicity (ADCC). Antibody binding to cell-associated siglec may induce complement-dependent cytotoxicity (CDC). The antibody may be a bispecific antibody. The antibody may include a CD3 binding domain. The antibody may include a chimeric antigen receptor. The antibody may include a conjugate. The conjugate may include a therapeutic agent. The therapeutic agent may include a toxin. The conjugate may include a detectable label.

In some embodiments, the present disclosure provides a method of treating a therapeutic indication in a subject using an antibody that binds a siglec and includes at least one human variable domain. The therapeutic indication may include a cancer-related indication. The cancer-related indication may include leukemia. The leukemia may include AML and/or CML. The AML and/or CML may include cancer cells expressing siglec12. The antibody may be administered to the subject. The antibody may be administered to the subject by injection or infusion.

In some embodiments, the present disclosure provides a method of detecting a siglec in a subject or a subject sample by contacting the subject or subject sample with an antibody that binds a siglec and includes at least one human variable domain. The antibody may include a detectable label. The antibody may bind to the siglec in the subject or subject sample and a detection reagent may be used to detect the bound antibody. The method may include detecting the siglec in a subject sample that includes a cell. The siglec may be detected in the subject sample by fluorescence-associated cell sorting (FACS) analysis. The siglec may be detected in the subject sample by immunohistochemistry. The immunohistochemistry may involve a colorimetric-based system or an immunofluorescence-based system for siglec detection.

In some embodiments, the present disclosure provides a method of stratifying subjects based on detection of a siglec in the subject or a subject sample by detecting a siglec in the subject or the subject sample according to any of the methods described herein and classifying the subject according to the type and/or level of siglec detected. The subject may be classified according to the presence or absence of siglec12 and/or level of siglec12 in the subject or the subject sample. The subject may be further classified according to the presence or absence of a specific siglec12 extracellular subdomain and/or level of the specific siglec12 extracellular subdomain in the subject or the subject sample.

In some embodiments, the present disclosure provides a method of treating a subject with cancer by administering an anti-siglec12 antibody to the subject, the anti-siglec12 antibody including: a heavy chain variable domain (VH) including an amino acid sequence selected from the group consisting of SEQ ID NOs: 1-7; and a light chain variable domain (VL) including an amino acid sequence selected from the group consisting of SEQ ID NOs: 8-14. The anti-siglec12 antibody may bind a subject cancer cell. The subject cancer cell may be an AML cancer cell. The anti-siglec12 antibody may be internalized by the subject cancer cell. The anti-siglec12 antibody may be conjugated to a cytotoxin. The cytotoxin may include calicheamicin. The cytotoxin may be MYLOTARG®. The VH may include the amino acid sequence of SEQ ID NO: 4 and the VL may include the amino acid sequence of SEQ ID NO: 11. The anti-siglec12 antibody may include a format selected from the group consisting of a Fab format and a whole antibody format. The equilibrium dissociation constant (Kd) for binding of the anti-siglec12 antibody to siglec12 may be from about 1 nM to about 10 nM.

### BRIEF DESCRIPTION OF THE FIGURES

The foregoing and other objects, features and advantages of particular embodiments of the disclosure will be apparent from the following description and illustrations in the accompanying figures.

FIG. 1 is a graph showing differential expression levels of siglec12 mRNA between normal tissue and tumors originating in the same tissue. RNA-seq measured gene expression is expressed in RSEM units (RNA-sequencing by Expectation Maximization).

FIG. 2 is a graph showing siglec12 mRNA transcript levels in various cancer cell lines measured by RNA-seq. mRNA level is expressed in TPM (Transcript Per kilobase Million).

FIG. 3 is a graph showing median siglec12 mRNA expression levels in various hematopoietic and lymphoid tissue cancer cell lines. RNA-seq measured gene expression is expressed as TPM.

FIG. 4 is a graph showing mRNA expression levels of different acute myeloid leukemia cell antigens in acute myeloid leukemia cell lines. RNA-seq measured gene expression is expressed in TPM.

FIG. 5 is a graph showing mRNA expression levels of different acute myeloid leukemia cell antigens in hematopoietic cells. RNA-seq measured gene expression is expressed in TPM.

FIG. 6 is a graph showing mRNA expression levels of different acute myeloid leukemia cell antigens in hematopoietic progenitor and stem cells. RNA-seq measured gene expression is expressed in TPM.

FIG. 7 is a graph comparing siglec12 and CD33 mRNA expression levels in various normal tissues. RNA-seq measured gene expression is expressed as median TPM.

### DETAILED DESCRIPTION

In some embodiments, the present disclosure provides antibodies that bind sialic acid binding-immunoglobulin type lectins (siglecs). Siglecs are a large family of proteins with fifteen members. They contain an N terminal V set immunoglobulin domain that binds to sialic acid glycans found on N-linked glycans and glycolipids (Duan and Paulson., 2020, Ann Rev Immunology 14, 365-395). The extracellular domains also consist of 1-16 C set immunoglobulin (Ig) domains. These Ig V set and C set domains are structurally similar to antibody variable and constant domains, respectively.

Siglec 1, 2, 4 and 15 are orthologues of one another. The remaining members of the siglec family are called CD33 related siglecs since they are thought to be derived from duplication of the CD33 gene. Sialic acids are nine carbon anionic sugars with a carboxyl group at C1, an N-acetyl group at C5 and a glycerol side chain from C7-C9. The sialic acid binding sites in the V set domains of siglecs contain a conserved arginine residue that forms a salt bridge with the C1 carboxyl group of sialic acid.

The majority of siglecs contain immunoreceptor tyrosine inhibitor motifs within their intracellular domain including ITIM or ITIM-like motifs that can participate in inhibitory or activating signaling. ITIMs are phosphorylated by src kinases and recruit src-homology 2 domain (SH2) containing phosphatases SHP-1 and SHP-2. SHP-1 and SHP-2 dephosphorylate the signaling molecules in the activation complex and subsequently participate in a variety of signaling pathways to influence cellular activation. SHP2 is a known proto-oncogene that has been shown to promote tumor cell invasion, prevent tumor cell apoptosis and promote cellular proliferation.

Siglec12 is comprised of two V set domains, two C set domains, a transmembrane domain, and two ITIM domains on the intracellular domain. Siglec12 is unique in the siglec family for several reasons: 1) the protein has two amino-terminal V-set domains (Yu et al., 2001, J Biol Chem, 276, 23816-24), compared with only one in all other siglecs; 2) there is a human-universal mutation of critical arginine residues in both V-set domains, rendering it unable to form a salt bridge with the carboxylic acid at C1 of sialic acid; 3) the Arg->Cys mutation of the V-set 1 domain is not present in orthologs of closely related “great apes” (chimpanzee, baboon, gorilla and orangutan) (Angata et al., 2001, J Biol Chem, 276, 40282-7); 4) the siglec12 gene harbors a common polymorphic frameshift mutation, causing early truncation of siglec12 and loss of expression of the long isoform (Mitra et al., 2011, J Biol Chem, 286, 23003-11). In normal tissues siglec12 is expressed in the spleen and more specifically, on tissue macrophages and monocytes. Unlike the other siglecs, siglec12 is expressed on certain epithelial cell cancers such as prostate cancer (Mitra et al., 2011, J Biol Chem, 286, 23003-11).

Siglec12 was originally cloned in 2001 and shown to have two isoforms derived from alternative splicing (Foussias et al., 2001, BBRC 284, 887-899). The spliced form removes the first exon generating a short isoform of 455 amino acids that lacks the first V set domain (Flores et al. 2019, Cell Mol Immunology, 16, 154-164). This is particularly relevant because the polymorphic frameshift mutation is in the first exon that causes premature termination of the protein. This frameshift mutation is present in 50-60% of the human population. However, because of the alternatively spliced isoform (short isoform), one or both isoforms is (are) expressed throughout the population so no null alleles exist in the population.

Similar to other siglecs, siglec12 has ITIM signaling domains intracellularly. Despite loss of its canonical sialic acid binding site, siglec12 has been demonstrated to recruit SHP1 and SHP2 to the proximal ITIM domain (Yu et al., J Biol Chem, 276, 23816-23824). While siglec12 in humans has lost the ability to form a salt bridge with the sialic acid carboxylic acid, it still retains key conserved residues that interact with sialic acids, namely two aromatic residues that form van der Waals contact with sialic acid in both V set domains (Tyr24 and Trp130) and the second V set domain (Tyr151 and Trp257). The sialic acid dependent binding data to date is controversial and based on sialic acid dependent red blood cell rosetting (Yu et al., J Biol Chem, 276, 23816-23824, Angata et al., 2001, J Biol Chem, 276, 40282-7).

### I. Compounds and Compositions

In some embodiments, the present disclosure provides compositions that interact with cell surface proteins. Such compositions may be antibodies that bind siglecs, referred to herein as “anti-siglec antibodies.” Anti-siglec antibodies may be useful for treating and/or diagnosing subjects, as well as other applications described herein.

### Antibodies

In some embodiments, the present disclosure provides antibodies (e.g., anti-siglec antibodies). As used herein, the term “antibody” is used in the broadest sense and specifically covers numerous embodiments, including, but not limited to, polyclonal antibodies, monoclonal antibodies, multispecific antibodies (e.g., bispecific antibodies or trispecific antibodies), human antibodies, humanized antibodies, chimeric antibodies, single chain antibodies, single chain Fv (scFv) formats, diabodies, intrabodies, unibodies, maxibodies, chimeric antigen receptors (CARs), and antibody fragments. As used herein the term “antibody fragment” refers to a portion of a whole antibody or a fusion protein that includes such a portion. Antibody fragments may include antigen binding regions. In some embodiments, antibody fragments include, but are not limited to Fab fragments, Fab' fragments, F(ab')₂ fragments, Fv fragments, Fc fragments, variable domains, constant domains, heavy chains, and light chains. In some embodiments, antibody fragments may be prepared by enzymatic digestion. Fab fragments may be prepared by papain digestion of whole antibodies. F(ab')₂ fragments may be prepared by pepsin treatment of whole antibodies.

“Native antibodies” refer to heterotetrameric glycoproteins having two identical light (L) chains and two identical heavy (H) chains. Genes encoding antibody heavy and light chains have been well characterized (see Matsuda, F. et al., 1998. The Journal of Experimental Medicine. 188(11); 2151-62 and Li, A. et al., 2004. Blood. 103(12): 4602-9, the content of each of which are herein incorporated by reference in their entirety). Light chains are linked to heavy chains by a covalent disulfide bond, while the number of disulfide linkages between heavy chains differs among immunoglobulin isotypes. Each heavy chain includes a variable domain (VH) followed by a number of constant domains. Light chains include a variable domain (VL) at one end and a constant domain at the other.

As used herein, the term “variable domain” refers to specific antibody domains found on both the antibody heavy and light chains that differ extensively in sequence among antibodies and determine antibody specificity for particular antigens. Variable domains include hypervariable regions that include amino acid residues responsible for antigen binding. Amino acids present within hypervariable regions determine the structure of complementarity determining regions (CDRs) that become part of the antigen-binding site of the antibody. As used herein, the term “CDR” refers to an antibody region having a structure that is complimentary to its target antigen or epitope. Other portions of variable domains, not interacting with antigen, are referred to as “framework regions” (FRs). Antigen-binding sites (also known paratopes) include amino acid residues necessary for interacting with particular antigens. Residues making up antigen-binding sites may be determined by CDR analysis. As used herein, “CDR analysis” refers to a process for determining which antibody variable domain amino acids form CDRs. Various methods of determining CDR sequences are known in the art and may be applied to known antibody sequences (Strohl, W.R. Therapeutic Antibody Engineering. Woodhead Publishing, Philadelphia PA. 2012. Ch. 3, p47-54, the contents of which are herein incorporated by reference in their entirety). CDR analysis may be carried out by co-crystallography with bound antigen. In some embodiments, CDR analysis includes computational assessments based on alignment with other antibodies (Strohl, W.R. Therapeutic Antibody Engineering. Woodhead Publishing, Philadelphia PA. 2012. Ch. 3, p47-54, the contents of which are herein incorporated by reference in their entirety). CDR analysis may utilize numbering schemes including, but not limited to, those described by Kabat [Wu, T.T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989), and Al-Lazikani, B. et al., 1997, J. Mol. Biol. 273(4):927-48, the contents of each of which are herein incorporated by reference in their entirety], Lefranc (Lefranc, M.P. et al., 2005, Immunome Res. 1:3), and Honegger (Honegger, A. and Pluckthun, A. 2001. J. Mol. Biol. 309(3):657-70, the contents of which are herein incorporated by reference in their entirety).

VH and VL domains each include three CDRs. VL CDRs are referred to herein as CDRL1, CDRL2 and CDRL3, in order of occurrence along the VL from N- to C-terminus. VH CDRs are referred to herein as CDRH1, CDRH2 and CDRH3, in order of occurrence along the VH from N- to C-terminus. Most CDRs have favored canonical structures except CDRH3, which includes amino acid sequences with high variability in sequence and length among antibodies resulting in varying three-dimensional antigen-binding domain structures (Nikoloudis, D. et al., 2014. PeerJ. 2:e456).

VH and VL domains have four framework regions (FRs) each positioned before, after, and between CDR regions. VH framework regions are referred to herein as FRH1, FRH2, FRH3, and FRH4 and VL framework regions are referred to herein as FRL1, FRL2, FRL3, and FRL4. FRs and CDRs of VH domains are typically in the order of FRH1-CDRH1-FRH2-CDRH2-FRH3-CDRH3-FRH4, from N- to C-terminus. FRs and CDRs of VL domains are typically in the order of FRL1-CDRL1-FRL2-CDRL2-FRL3-CDRL3-FRL4, from N- to C-terminus.

“Fv” antibody fragments include the minimum antibody fragment needed to form a complete antigen-binding site. These regions include a heavy chain and light chain variable domain dimer in tight, non-covalent association. Stable Fv fragments may be synthesized recombinantly through incorporation of a flexible linker between light and heavy chain variable domains to form single chain Fv (scFv) formats. Other Fv formats may include a disulfide bridge between heavy and light chain variable domains (Strohl, W.R. Therapeutic Antibody Engineering. Woodhead Publishing, Philadelphia PA. 2012. Ch. 3, p46-47, the contents of which are herein incorporated by reference in their entirety).

Vertebrate antibody light chains typically fall into one of two distinct types, kappa and lambda based on constant domain amino acid sequence. Additional antibody classes depend on heavy chain constant domain amino acid sequences.

ScFvs may be utilized in conjunction with phage display, yeast display or other display technologies to create scFv libraries expressed in association with cell or coat surfaces (e.g. in association with phage coat proteins) for identification of peptides with high antigen affinity. Antibodies may be prepared as scFvFc antibodies which include fusions of scFvs with antibody Fc domains.

“Chimeric antibodies” refer to antibodies with portions derived from two or more sources, e.g., from different species. Chimeric antibodies may include mouse variable domains and human constant domains. Examples of chimeric antibodies and related methods of synthesis are described in U.S. Pat. Nos. 5,807,715; 4,816,567; and 4,816,397, the contents of each of which are incorporated herein by reference in their entirety.

“Chimeric antigen receptors” or “CARs” refer to artificial receptors engineered for expression on immune effector cell surfaces facilitating specific targeting of such immune effector cells to cells expressing CAR target antigens. CARs may be designed to include one or more antibody segments, e.g., antibody variable domains and/or antibody CDRs, to direct immune effector cells to antigens recognized by such antibody segments. In some cases, CARs are designed to specifically target cancer cells, leading to immune-mediated clearance of the cancer cells.

The term “monoclonal antibody” refers to an antibody obtained from a substantially homogeneous cell population producing substantially identical antibodies binding to the same epitope of a specific target antigen. “Polyclonal” antibody preparations typically include a heterogeneous group of antibodies directed against different epitopes of a specific target.

Monoclonal antibodies may be prepared using a number of different methods. Monoclonal antibodies may be chimeric with portions derived from two or more species. For example, chimeric monoclonal antibodies may include antibody variable domains corresponding with human variable domain sequences and antibody constant domains corresponding with mouse constant domain sequences.

Antibodies of the present disclosure may be derived from or correspond with antibodies of different animal origins including mammals, birds, reptiles, and insects. Mammalian antibodies may be, for example, of murine (e.g., mouse or rat), rabbit, donkey, sheep, goat, guinea pig, camel, bovine, horse, or human origin.

As used herein, the term “antibody variant” refers to a biomolecule resembling an antibody in structure, sequence and/or function, but including some differences in their amino acid sequence, composition or structure as compared to another antibody or a native antibody.

Antibodies of the present disclosure may include a conjugate. As used herein, the term “conjugate” refers to any agent, cargo, or chemical moiety attached to a recipient compound or the process of attaching such an agent, cargo, or chemical moiety. As used herein, the term “antibody conjugate” refers to an antibody having an attached agent, cargo, or chemical moiety. Conjugates may include therapeutic agents. Therapeutic agents may include drugs. Antibody conjugates that include conjugated drugs are referred to herein as “antibody drug conjugates.” Antibody drug conjugates may be used to deliver conjugated drugs to specific targets based on antibody affinity for specific proteins or epitopes. Antibody drug conjugates may be used to focus biological activity of conjugated drugs to targeted cells, tissues, organs, etc.

In some embodiments, antibody conjugates include detectable labels. Detectable labels may be used to detect antibody binding. Examples of detectable labels include, but are not limited to, radioisotopes, fluorophores, chromophores, chemiluminescent compounds, enzymes, enzyme co-factors, dyes, metal ions, ligands, biotin, avidin, streptavidin, haptens, quantum dots, or any other detectable labels known in the art or described herein.

Conjugates may be attached directly or via a linker. Direct attachment may involve covalent bonding or non-covalent associations (e.g., ionic bonds, hydrostatic bonds, hydrophobic bonds, hydrogen bonds, hybridization, etc.). Linkers used for conjugation may include any chemical structures capable of joining antibodies with conjugates. For example, linkers may include polymeric molecules (e.g., nucleic acids, polypeptides, polyethylene glycols, carbohydrates, lipids, or combinations thereof). Antibody conjugate linkers may be cleavable (e.g., through contact with an enzyme, change in pH, or change in temperature).

### Antigens

Antibodies may be developed (e.g., through immunization) or selected (e.g., from pool of candidates), for example, using antigens. An “antigen,” as referred to herein, is any entity that induces an immune response in an organism or may simply refer to an antibody binding partner. Immune responses are reactions of cells, tissues and/or organs of an organism to a foreign entity. Immune responses typically lead to the production of one or more antibodies against a foreign entity by an organism.

In some embodiments, antigens of the present disclosure include siglecs or portions thereof, referred to herein as “siglec antigens.” Siglec antigens may include siglec extracellular domains. Siglec antigens may include fusion proteins of siglecs or siglec portions with other entities. In some embodiments, siglec antigens include fusion proteins of siglec extracellular domains with antibody Fc regions. Fc regions used in siglec antigen fusion proteins may be derived from different species.

As used herein, the term “target antigen” refers to an entity, protein, or epitope to which an antibody binds or for which an antibody is desired, designed, or developed to have affinity for.

### Immunization

In some embodiments, antibodies may be prepared by immunization. Immunization may include immunizing a host with the target antigen of an antibody being developed. Host animals (e.g., mice, rabbits, goats, primates, or llamas) may be utilized for immunizations to elicit lymphocytes specific for a target antigen. Lymphocytes may be isolated and fused with immortalized cell lines to yield hybridomas which can be cultured and propagated (e.g., see Kohler, G. et al., Continuous cultures of fused cells secreting antibody of predefined specificity. Nature. 1975 Aug. 7;256(5517):495-7, the contents of which are herein incorporated by reference in their entirety). In some embodiments, lymphocyte immunization may be carried out in vitro.

Immortalized cell lines used for hybridoma generation may be transformed mammalian cells (e.g., myeloma cells of rodent, rabbit, bovine, or human origin). Rat or mouse myeloma cell lines may be employed. Hybridoma cells may be cultured with one or more substances that inhibit growth or survival of unfused cells. In some embodiments, culture media supplemented with hypoxanthine, aminopterin, and thymidine (“HAT medium”) may be used to culture hybridoma cells generated from parental cells lacking hypoxanthine guanine phosphoribosyl transferase (HGPRT or HPRT) to prevent growth of HGPRT-deficient (unfused) cells.

In some embodiments, immortalized cell lines are murine myeloma lines. Human myeloma and mouse-human heteromyeloma cell lines may be used for preparation of human monoclonal antibodies (e.g., see Kozbor, D. et al., A human hybrid myeloma for production of human monoclonal antibodies. J Immunol. 1984 December; 133(6):3001-5 and Brodeur, B. et al., Monoclonal Antibody Production Techniques and Applications. Marcel Dekker, Inc., New York. 1987; 33:51-63, the contents of each of which are herein incorporated by reference in their entireties).

Media from hybridoma cell cultures may be assayed for secreted antibodies with desired specificity. Such assays may include, but are not limited to, enzyme-linked immunosorbent assays (ELISA) and fluorescence-associated cell sorting (FACS) assays. Assays may be carried out to determine binding specificity for target antigens. Hybridoma cells producing antibodies with desirable characteristics may be subcloned and expanded using standard methods. Hybridoma antibodies may be isolated and purified using standard immunoglobulin purification procedures, e.g., protein A-SEPHAROSE® purification, hydroxyapatite chromatography, gel electrophoresis, dialysis, or affinity chromatography. Hybridoma cells may be grown in vivo in mammals as ascites. In some embodiments, antibodies may be isolated directly from host serum samples.

In some embodiments, antibodies generated through immunization may be analyzed for amino acid sequence or encoding nucleic acid sequence for reproduction using recombinant technology. For example, antibodies may be produced by insertion of nucleic acid sequences encoding antibodies into expression vectors for introduction into cells and expression of encoded antibodies.

### Anti-Siglec Antibodies

In some embodiments, the present disclosure provides anti-siglec antibodies. The antibodies may bind to siglec12. In some embodiments, anti-siglec antibodies of the present disclosure include any of the antibodies listed in Table 1.

TABLE 1
Anti-siglec antibodies
ID#

S0001
S0002
S0003
S0004
S0005
S0006
S0007
S0008
S0009
S0010
S0011
S0012
S0013
S0014
S0015
S0016
S0017
S0018
S0019
S0020
S0021
S0022
S0023
S0024
S0025
S0026
S0027
S0028
S0029
S0030
S0031
S0032
S0033
S0034
S0035
S0036
S0037
S0038
S0039
S0040
S0041
S0042
S0043
S0044
S0045
S0046
S0047
S0048
S0049
S0050
S0051
S0052
S0053
S0054
S0055
S0056
S0057
S0058
S0059
S0060
S0061
S0062
S0063
S0064
S0065
S0066
S0067
S0068
S0069
S0070
S0071
S0072
S0073
S0074
S0075
S0076
S0077
S0078
S0079
S0080
S0081
S0082
S0083
S0084
S0085
S0086
S0087
S0088
S0089
S0090
S0091
S0092
S0093
S0094
S0095
S0096
S0097
S0098
S0099
S0100
S0101
S0102
S0103
S0104
S0105
S0106
S0107
S0108
S0109
S0110
S0111
S0112
S0113
S0114

Antibodies may bind siglec12 extracellular domains. Such siglec12 extracellular domains may include long isoforms or short isoforms. In some embodiments, antibodies of the present disclosure bind a subdomain of the siglec12 extracellular domain (long or short isoform). Such subdomains may include the siglec12 Vset1 (S12-V1) region, the Vset2 (S12-V2) region, the C2set1 (S12-C1) region, or the C2set2 (S12-C2) region. Antibodies may bind to combinations of subdomains. Combinations may include S12-V2 and S12-C1 (S12-V2C1). In some embodiments, combinations may include S12-V2, S12-C1, and S12-C2 (S12-V2C1C2), which correspond with siglec12 short isoform extracellular domains.

Anti-siglec antibodies binding to S12-V2C1 may be used to target and/or detect both long and short siglec12 isoforms. Such antibodies may be useful for targeting cells expressing both long and short siglec12 isoforms. In some embodiments, antibodies binding to S12-V2C1 may not bind to S12-V1 or S12-C2 or may bind to either with reduced affinity in comparison to affinity for S12-V2C1.

In some embodiments, antibodies of the present disclosure bind to S12-C2. S12-C2 is located on a region of the siglec12 extracellular domain that is closer to the cell surface. Binding to epitopes close to the cell surface has been shown to provide greater sensitivity with respect to cell cytotoxicity with anti-CD3 bispecific antibody treatments (e.g., see Estey E.H. et al., Am J Hematol. 2018. 93:1267-91). Accordingly, anti-siglec antibodies described herein that bind to S12-C2 may be utilized to produce bispecific antibodies with both a siglec binding region and a CD3 binding region.

In some embodiments, antibodies binding to siglec12 may not bind to other siglecs, including other siglecs of the CD33-related siglec family (e.g., siglec7 or siglec9, which have the greatest sequence identity to siglec12).

Anti-siglec antibodies may bind siglecs associated with cells (e.g., cell surfaces). Such cells may include cancer cells. Cancer cells bound by anti-siglec antibodies (e.g., anti-siglec12 antibodies) may be leukemia cells. Such leukemia cells may include, but are not limited to, acute myeloid leukemia (AML) cells and chronic myeloid leukemia (CML) cells.

In some embodiments, anti-siglec antibodies of the present disclosure may include at least one human antibody variable domain. Such antibodies may be prepared through immunization of hosts expressing antibody variable domains corresponding with human antibody variable domains. Hosts expressing variable domains corresponding with human variable domains may include Trianni transgenic mice (e.g., see United States Publication Number US2013/0219535, the contents of which are herein incorporated by reference in their entirety). Endogenous immunoglobulin VH, DH and JH; Vκ and Jκ; and Vλ and Jλ; gene segments have been replaced in these mice by the full repertoire of human counterpart gene segments. Human gene coding sequences are flanked by mouse cis-acting regulatory elements and recombination signal sequences allowing for a “normal” immune response, including isotype switching with heavy and light chain affinity maturation. The resulting human-mouse chimera antibodies contain human antibody variable domains flanked by mouse heavy and light chain immunoglobulin constant regions. These antibodies are more readily converted to fully human antibodies using standard methods to combine the human variable domains with human constant regions.

In some embodiments, anti-siglec antibodies of the present disclosure induce cell death upon binding to cell-associated siglecs. Such antibodies may induce antibody-dependent cellular cytotoxicity (ADCC) upon binding to siglec-associated cells. Antibody binding to cell-associated siglecs may induce complement-dependent cytotoxicity (CDC).

In some embodiments, anti-siglec antibodies of the present disclosure may inhibit tumor immunosuppression upon binding to cell-associated siglecs. Tumor immunosuppression involves dysfunctional suppression of anti-tumor immune responses. Anti-siglec antibodies may target cells involved in anti-tumor immunosuppression and inhibit their suppressive activities.

In some embodiments, anti-siglec antibodies (e.g., anti-siglec12 antibodies) of the present disclosure may be used to develop or be incorporated into bispecific antibodies. Such antibodies may include a CD3 binding domain to recruit CD3-expressing immune cells to cells expressing siglecs (e.g., siglec12).

In some embodiments, anti-siglec antibodies (e.g., anti-siglec12 antibodies) of the present disclosure may be used to develop or be incorporated into chimeric antigen receptors (CARs). Such CARs may be expressed on the surface of immune cells and facilitate recruitment of the immune cells to cells expressing siglecs (e.g., siglec12).

In some embodiments, anti-siglec antibodies (e.g., anti-siglec12 antibodies) of the present disclosure may include conjugates. Conjugates may include therapeutic agents. In some embodiments, therapeutic agents may include toxins. Such toxins may include, but are not limited to, radioisotopes, saporins, taxanes, vinca alkaloids, anthracyclines, calicheamicins, duocarmycins, pyrrolobenzodiazepine dimers, and platinum-based agents. Calicheamicins may include MYLOTARG®. In some embodiments, conjugates may include detectable labels.

Anti-siglec antibodies of the present disclosure may include variable domain amino acid sequences according to any of those listed in Table 2. In some embodiments, anti-siglec antibody variable domains include fragments or variants of variable domain amino acid sequences listed. Such fragments or variants may include from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the variable domain amino acid sequences listed in Table 2.

TABLE 2
Variable domain amino acid sequences

| | | | SEQ |
|---|---|---|---|
| ID# | Region | Sequence | ID NO |
| S0023 | VH | EVQLLESGGGLVQPGGSLRLSCAASGFTFST | 1 |
| | | FAMSWVRQAPGKGLEWVSTISGGGDSTYY | |
| | | ADSVKGRFTISRDNSKNMMSLQMNSLRAV | |
| | | DTAVYYCVKGGGDEYFYYGMDVWGQGTT | |
| | | VTVSS | |
| S0040 | VH | QVQLVQSGAEVKKPGASVKVSCKASGYTF | 2 |
| | | TSYDINWVRQATGQGLEWMGWMNPNSGN | |
| | | TGYAQNFQGRVTMTRNTSINTAYMELSSLR | |
| | | SEDTAVYYCTRGGPTLVRGVIIRDAFDFWG | |
| | | QGTMVTVSS | |
| S0042 | VH | QVQLVQSGTEVKKPGASVKVSCKVSGYTL | 3 |
| | | SDLSIHWVRQAPGKGLEWMGGFEPEDGEII | |
| | | YAQKFQGRVIMTEDTSTDTAYMKLSSLRSE | |
| | | DTAVYYCATDKRYFDWLLSGFDYWGQGT | |
| | | LVTVSS | |
| S0057 | VH | QGQLVQSGAEVKKPGASVKVSCKASGYTF | 4 |
| | | NNYGISWVRQAPGQGLEWMGWISAYNGFT | |
| | | RSTQKFQGRVTLTTDTSTSTAHMELRSLRS | |
| | | DDTAAYYCARDGGDGFDPWGQGTLVTVSS | |
| S0102 | VH | QVQLVESGGGVVQPGRSLRLSCAASGFTFS | 5 |
| | | SYGMHWVRQAPGKGLEWVAVISYDGSNK | |
| | | YYTDSVKGRFTISRDNSKNTLYLQMNSLRA | |
| | | EDTAVYYCARGGYYGSGSLDYWGQGTLV | |
| | | TVSS | |
| S0103 | VH | QVQLVQSGAEVKKPGASVKVSCKASGYTF | 6 |
| | | TSYDINWVRQATGQGLEWMGWMNPNSGN | |
| | | TGYAQNFQGRVTMTRDTSVNTAYMELSSL | |
| | | RSEDTAVYYCARGGPNLVRGVIIRDAFDFW | |
| | | GQGTMVTVSS | |
| S0114 | VH | QVQLVESGGGVVQPGRSLRLSCAASGFTFSI | 7 |
| | | YGMHWVRQAPGKGLEWVAVISNDGSNEY | |
| | | YADSVKGRFTISRDYSKNTLYLQMNSLRAX | |
| | | DTAVYYCAREGESYPFDYWGQGTLVTVSS | |
| S0023 | VL | DIQMTQSPSSLSASVGDRVTITCRASQGIRN | 8 |
| | | DLGWYQQKPGKAPKRLIYSASSLQSGVPSR | |
| | | FSGSGSGTEFTLTISSLQPEDFATYYCLQHN | |
| | | SYPYTFGQGTPLDIKRA | |
| S0040 | VL | EIVMTQSPATLSVSPGERATLSCRASQSVSN | 9 |
| | | SFAWYQQKPGQAPRLLIYGAFTRATGIPAR | |
| | | FSGSGSGTEFTLTISSLQSEDFAVYYCQQYK | |
| | | TWPLTFGGGTKVEIKRA | |
| S0042 | VL | DIQLTQSPSFLSASVGDRVTITCRASQGISSY | 10 |
| | | LAWYQQKPGKAPKLLIFAASTLQSGVPSRF | |
| | | SGSGSGTEFTLTISSLQPEDFATYYCQRLHS | |
| | | YPRTFGQGTKVEIKRA | |
| S0057 | VL | EIVLTQSPATLSLSPGERATLSCRASQSVSSF | 11 |
| | | LAWYQQKPGQAPRLLIFDASNRATGIPARF | |
| | | SGSGSGTDFTLTISSLEPEDFAVYYCQQRSN | |
| | | WITFGQGTRLEIKRA | |
| S0102 | VL | DVVMTQSPLSLPVTLGQPASISCRSSQSLVY | 12 |
| | | SDGNTYLNWFQQRPGQSPRRLIYKVSNRDS | |
| | | GVPDRFSGSGSGTDFTLKISRVEAEDVGVY | |
| | | YCMQGTHWPWTFGQGTKVEIKRA | |
| S0103 | VL | EIVMTQSPATLSVSPGERATLSCRASQSVSN | 13 |
| | | SFAWYQQKPGQAPRLLIYGAFTRATGIPAR | |
| | | FSGSGSGIEFTLTISSLQSEDFAVYYCQQYK | |
| | | TWPLTFGGGTKVEIKRA | |
| S0114 | VL | EIVMTQSPATLSVSPGERATLSCRASQSVSS | 14 |
| | | NLAWYQQKPGQAPRLLIYGAFFRATGIPAR | |
| | | FSGSGSGTEFTLTISSLQSEDFAVYYCQQYN | |
| | | NWPYTFGQGTKLEIKRA | |

Anti-siglec antibody variable domains of the present disclosure may be encoded by nucleic acid sequences listed in Table 3. In some embodiments, nucleic acid sequences encoding anti-siglec antibody variable domains of the present disclosure may include fragments or variants of the nucleic acid sequences listed. Such fragments or variants may include from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the nucleic acid sequences listed. In some embodiments, nucleic acid sequences encoding anti-siglec antibody variable domains of the present disclosure include codon-optimized variants of the nucleic acid sequences listed.

TABLE 3
Variable domain nucleic acid sequences

| ID# | Region | Sequence | SEQ ID NO |
|---|---|---|---|
| S0023 | VH | gaggtgcagctgttggagtctgggggaggcttggtacagccg | 15 |
| | | ggggggtccctgagactctcctgtgcagcctctggattcacctt | |
| | | tagtacctttgccatgagctgggtccgccaggctccaggaaa | |
| | | ggggctggagtgggtctcaactattagtggtggtggtgacagt | |
| | | acatactacgcagactccgtgaagggccggttcaccatctcca | |
| | | gagacaattccaagaacatgatgtctctacaaatgaacagcct | |
| | | gagagccgtggacacggccgtatattactgtgtgaaggggg | |
| | | ggggagacgaatacttctactacggtatggacgtctggggcc | |
| | | aagggaccacggtcaccgtctcctca | |
| S0040 | VH | caggtacagctggtgcagtctggggctgaggtgaagaagcct | 16 |
| | | ggggcctcagtgaaggtctcctgcaaggcttctggatacacct | |
| | | tcaccagttatgatatcaactgggtgcgacaggccactggaca | |
| | | agggcttgagtggatgggatggatgaaccctaacagtggtaa | |
| | | cacaggctatgcacagaatttccagggcagagtcaccatgac | |
| | | caggaacacctccattaacacagcctacatggagctgagcag | |
| | | cctgagatctgaggacacggccgtgtattactgtacgagagg | |
| | | ggggcctactctggttcggggagttattataagagatgcttttg | |
| | | atttctggggccaagggacaatggtcaccgtctcctca | |
| S0042 | VH | caggtccagttggtccagtctgggactgaggtgaagaagccg | 17 |
| | | ggggcctcagtgaaggtctcctgcaaagtttccggatacaccc | |
| | | tcagtgacttatccattcactgggtgcgacaggctcctggaaa | |
| | | agggcttgagtggatgggaggttttgaacctgaagatggtgaa | |
| | | ataatctacgcacagaagttccagggcagagtcattatgaccg | |
| | | aggacacatctacagacacagcctacatgaaactgagcagcc | |
| | | tgagatctgaggacacggccgtgtattattgtgcaacagataa | |
| | | acgatattttgactggttattaagcggctttgactattggggcca | |
| | | gggaaccctggtcaccgtctcctca | |
| S0057 | VH | cagggtcagctggtgcagtctggagctgaggtgaagaagcct | 18 |
| | | ggggcctcagtgaaggtctcctgcaaggcttctggttacacctt | |
| | | taacaactatggtatcagttgggtgcgacaggcccctggacaa | |
| | | gggcttgagtggatgggatggatcagcgcttataatggtttcac | |
| | | aagaagtacacagaaattccagggcagagtcaccttgaccac | |
| | | agacacatcaacgagcacagcccacatggagttgaggagcc | |
| | | tgagatctgacgacacggccgcttattattgtgcgagagatgg | |
| | | gggagacgggttcgacccctggggccagggaaccctggtc | |
| | | accgtctcctca | |
| S0102 | VH | caggtgcagctggtggagtctgggggaggcgtggtccagcc | 19 |
| | | tgggaggtccctgagactctcctgtgcagcctctggattcacct | |
| | | tcagtagctatggcatgcactgggtccgccaggctccaggca | |
| | | aggggctggagtgggtggcagttatatcatatgatggaagtaa | |
| | | taaatactatacagactccgtgaagggccgattcaccatctcca | |
| | | gagacaattccaagaacacgctgtatctgcaaatgaacagcct | |
| | | gagagctgaggacacggctgtgtattactgtgcgagagggg | |
| | | gttactatggttcggggagccttgactactggggccagggaa | |
| | | ccctggtcaccgtctcctca | |
| S0103 | VH | caggtgcagctggtgcagtctggggctgaggtgaagaagcc | 20 |
| | | tggggcctcagtgaaggtctcctgcaaggcttctggatacacc | |
| | | ttcaccagttatgatatcaactgggtgcgacaggccactggac | |
| | | aagggcttgagtggatgggatggatgaaccctaacagtggta | |
| | | acacaggctatgcacagaatttccagggcagagtcaccatga | |
| | | ccagggacacctccgtaaacacagcctacatggagctgagc | |
| | | agcctgagatctgaggacacggccgtgtattactgtgcgaga | |
| | | ggggggcctaatctggttcggggagttattataagagatgcttt | |
| | | tgatttctggggccaagggacaatggtcaccgtctcctca | |
| S0114 | VH | caggtgcaactggtggagtctgggggaggcgtggtccagcc | 21 |
| | | tgggaggtccctgagactctcctgtgcagcctctggattcacct | |
| | | tcagtatctatggcatgcactgggtccgccaggctccaggcaa | |
| | | ggggctggagtgggtggcagttatatcaaatgatggaagtaat | |
| | | gaatactatgcagactccgtgaagggccgattcaccatctcca | |
| | | gagactattccaagaacacgctgtatctgcaaatgaacagcct | |
| | | gagagctgangacacggctgtgtattattgtgcgagagaggg | |
| | | ggagagctacccctttgactactggggccagggaaccctggt | |
| | | caccgtctcctca | |
| S0023 | VL | gacatccagatgacccagtctccatcctccctgtctgcatctgt | 22 |
| | | aggagacagagtcaccatcacttgccgggcaagtcagggca | |
| | | ttagaaatgatttaggctggtatcagcagaaaccagggaaagc | |
| | | ccctaagcgcctgatctattctgcatccagtttgcaaagtgggg | |
| | | tcccatcaaggttcagcggcagtggatctgggacagaattcac | |
| | | tctcacaatcagcagcctgcagcctgaagattttgcaacttatta | |
| | | ctgtctacagcataatagttacccgtacacttttggccagggga | |
| | | ccccgctggacatcaaacgggct | |
| S0040 | VL | gaaatagtgatgacgcagtctccagccaccctgtctgtgtctcc | 23 |
| | | aggggaaagagccaccctctcctgcagggccagtcagagtg | |
| | | ttagcaacagctttgcctggtaccagcagaaacctggccagg | |
| | | ctcccaggctcctcatctatggtgcattcaccagggccactggt | |
| | | atcccagccaggttcagtggcagtgggtctgggacagagttc | |
| | | actctcaccatcagcagcctgcagtctgaagattttgcagtttat | |
| | | tactgtcagcagtataagacctggcctctcactttcggcggag | |
| | | ggaccaaggtggagatcaaacgggct | |
| S0042 | VL | gacatccagttgacccagtctccatccttcctgtctgcatctgta | 24 |
| | | ggagacagagtcaccatcacttgccgggccagtcagggcatt | |
| | | agcagttatttagcctggtatcagcaaaaaccagggaaagcc | |
| | | cctaagctcctgatctttgctgcatccactttgcaaagtggggtc | |
| | | ccatcaaggttcagcggcagtggatctgggacagaattcactc | |
| | | tcacaatcagcagcctgcagcctgaggattttgcaacttattact | |
| | | gtcaacgacttcatagttaccctcggacgttcggccaagggac | |
| | | caaggtggaaatcaaacgggct | |
| S0057 | VL | gaaattgtgttgacacagtctccagccaccctgtctttgtctcca | 25 |
| | | ggggaaagagccaccctctcctgcagggccagtcagagtgtt | |
| | | agcagcttcttagcctggtaccaacagaaacctggccaggct | |
| | | cccaggctcctcatctttgatgcatccaacagggccactggca | |
| | | tcccagccaggttcagtggcagtgggtctgggacagacttca | |
| | | ctctcaccatcagcagcctagagcctgaagattttgcagtttatt | |
| | | actgtcagcagcgtagcaactggatcaccttcggccaaggga | |
| | | cacgactggagattaaacgggctg | |
| S0102 | VL | gatgttgtgatgactcagtctccactctccctgcccgtcaccctt | 26 |
| | | ggacagccggcctccatctcctgcaggtctagtcaaagcctc | |
| | | gtatacagtgatggaaacacctacttgaattggtttcagcagag | |
| | | gccaggccaatctccaaggcgcctaatttataaggtttctaacc | |
| | | gggactctggggtcccagacagattcagcggcagtgggtca | |
| | | ggcactgatttcacactgaaaatcagcagggtggaggctgag | |
| | | gatgttggggtttattactgcatgcaaggtacacactggccgtg | |
| | | gacgttcggccaagggaccaaggtggaaatcaaacgggct | |
| S0103 | VL | gaaatagtgatgacgcagtctccagccaccctgtctgtgtctcc | 27 |
| | | aggggaaagagccaccctctcctgcagggccagtcagagtg | |
| | | ttagcaacagctttgcctggtaccagcagaaacctggccagg | |
| | | ctcccaggctcctcatctatggtgcattcaccagggccactggt | |
| | | atcccagccaggttcagtggcagtgggtctgggatagagttca | |
| | | ctctcaccatcagcagcctgcagtctgaagattttgcagtttatt | |
| | | actgtcagcagtataagacctggcctctcactttcggcggagg | |
| | | gaccaaggtggagatcaaacgggct | |
| S0114 | VL | gaaatagtgatgacgcagtctccagccaccctgtctgtgtctcc | 28 |
| | | aggggaaagagccaccctctcctgcagggccagtcagagtg | |
| | | ttagcagcaacttagcctggtaccagcagaaacctggccagg | |
| | | ctcccaggctcctcatctatggtgcattttttagggccactggta | |
| | | tcccagccaggttcagtggcagtgggtctgggacagagttca | |
| | | ctctcaccatcagcagcctgcagtctgaagattttgcagtttatt | |
| | | actgtcagcagtataataactggccgtacacttttggccaggg | |
| | | gaccaagctggagatcaaacgggct | |

In some embodiments, anti-siglec antibodies of the present disclosure include VH and VL domain pairs, wherein each member of the pair includes a variable domain amino acid sequence presented herein and/or is encoded by a variable domain nucleic acid sequence presented herein. In some embodiments, anti-siglec antibodies of the present disclosure include a variable domain pair according to any of those listed in Table 4.

TABLE 4
Variable domain pairs

| ID# | VH SEQ ID NO | VL SEQ ID NO |
|---|---|---|
| S0023 | 1 | 8 |
| S0040 | 2 | 9 |
| S0042 | 3 | 10 |
| S0057 | 4 | 11 |
| S0102 | 5 | 12 |
| S0103 | 6 | 13 |
| S0114 | 7 | 14 |

In some embodiments, anti-siglec antibodies of the present disclosure include one or more CDRs with amino acid sequences derived from one or more variable domain amino acid sequence provided in Table 2. In some embodiments, anti-siglec antibodies of the present disclosure include one or more CDRs encoded by nucleic acid sequences derived from one or more variable domain nucleic acid sequences provided in Table 3. Anti-siglec antibody CDRs may include one or more amino acid residues involved in antigen binding (e.g., as determined by co-crystallography with bound antigen). Anti-siglec antibodies of the present disclosure may include CDRs identified through CDR analysis of variable domain sequences presented herein via co-crystallography with bound antigen; by computational assessments based on comparisons with other antibodies (e.g., see Strohl, W.R. Therapeutic Antibody Engineering. Woodhead Publishing, Philadelphia PA. 2012. Ch. 3, p47-54); or Kabat, Chothia, Al-Lazikani, Lefranc, or Honegger numbering schemes, as described previously. In some embodiments, anti-siglec antibody CDR amino acid sequences may include any of those presented in Table 5, or fragments thereof. In some embodiments, anti-siglec antibodies of the present disclosure include CDRs that include amino acid sequence variants of those listed. Amino acid fragments or variants included in anti-siglec antibody CDRs may include from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the amino acid sequences listed.

TABLE 5
CDR amino acid sequences

| | | Numbering | | SEQ |
|---|---|---|---|---|
| ID# | Region | Scheme | Sequence | ID NO |
| S0023 | CDRH1 | Kabat | TFAMS | 29 |
| S0040 | CDRH1 | Kabat | SYDIN | 30 |
| S0042 | CDRH1 | Kabat | DLSIH | 31 |
| S0057 | CDRH1 | Kabat | NYGIS | 32 |
| S0102 | CDRH1 | Kabat | SYGMH | 33 |
| S0103 | CDRH1 | Kabat | SYDIN | 30 |
| S0114 | CDRH1 | Kabat | IYGMH | 34 |
| S0023 | CDRH2 | Kabat | TISGGGDSTYYADSVKG | 35 |
| S0040 | CDRH2 | Kabat | WMNPNSGNTGYAQNFQG | 36 |
| S0042 | CDRH2 | Kabat | GFEPEDGEIIYAQKFQG | 37 |
| S0057 | CDRH2 | Kabat | WISAYNGFTRSTQKFQG | 38 |
| S0102 | CDRH2 | Kabat | VISYDGSNKYYTDSVKG | 39 |
| S0103 | CDRH2 | Kabat | WMNPNSGNTGYAQNFQG | 36 |
| S0114 | CDRH2 | Kabat | VISNDGSNEYYADSVKG | 40 |
| S0023 | CDRH3 | Kabat | GGGDEYFYYGMDV | 41 |
| S0040 | CDRH3 | Kabat | GGPTLVRGVIIRDAFDF | 42 |
| S0042 | CDRH3 | Kabat | DKRYFDWLLSGFDY | 43 |
| S0057 | CDRH3 | Kabat | DGGDGFDP | 44 |
| S0102 | CDRH3 | Kabat | GGYYGSGSLDY | 45 |
| S0103 | CDRH3 | Kabat | GGPNLVRGVIIRDAFDF | 46 |
| S0114 | CDRH3 | Kabat | EGESYPFDY | 47 |
| S0023 | CDRL1 | Kabat | RASQGIRNDLG | 48 |
| S0040 | CDRL1 | Kabat | RASQSVSNSFA | 49 |
| S0042 | CDRL1 | Kabat | RASQGISSYLA | 50 |
| S0057 | CDRL1 | Kabat | RASQSVSSFLA | 51 |
| S0102 | CDRL1 | Kabat | RSSQSLVYSDGNTYLN | 52 |
| S0103 | CDRL1 | Kabat | RASQSVSNSFA | 49 |
| S0114 | CDRL1 | Kabat | RASQSVSSNLA | 53 |
| S0023 | CDRL2 | Kabat | SASSLQS | 54 |
| S0040 | CDRL2 | Kabat | GAFTRAT | 55 |
| S0042 | CDRL2 | Kabat | AASTLQS | 56 |
| S0057 | CDRL2 | Kabat | DASNRAT | 57 |
| S0102 | CDRL2 | Kabat | KVSNRDS | 58 |
| S0103 | CDRL2 | Kabat | GAFTRAT | 55 |
| S0114 | CDRL2 | Kabat | GAFFRAT | 59 |
| S0023 | CDRL3 | Kabat | LQHNSYPYT | 60 |
| S0040 | CDRL3 | Kabat | QQYKTWPLT | 61 |
| S0042 | CDRL3 | Kabat | QRLHSYPRT | 62 |
| S0057 | CDRL3 | Kabat | QQRSNWIT | 63 |
| S0102 | CDRL3 | Kabat | MQGTHWPWT | 64 |
| S0103 | CDRL3 | Kabat | QQYKTWPLT | 61 |
| S0114 | CDRL3 | Kabat | QQYNNWPYT | 65 |
| S0023 | CDRH1 | Lefranc | GFTFSTFA | 66 |
| S0040 | CDRH1 | Lefranc | GYTFTSYD | 67 |
| S0042 | CDRH1 | Lefranc | GYTLSDLS | 68 |
| S0057 | CDRH1 | Lefranc | GYTFNNYG | 69 |
| S0102 | CDRH1 | Lefranc | GFTFSSYG | 70 |
| S0103 | CDRH1 | Lefranc | GYTFTSYD | 67 |
| S0114 | CDRH1 | Lefranc | GFTFSIYG | 71 |
| S0023 | CDRH2 | Lefranc | ISGGGDST | 72 |
| S0040 | CDRH2 | Lefranc | MNPNSGNT | 73 |
| S0042 | CDRH2 | Lefranc | FEPEDGEI | 74 |
| S0057 | CDRH2 | Lefranc | ISAYNGFT | 75 |
| S0102 | CDRH2 | Lefranc | ISYDGSNK | 76 |
| S0103 | CDRH2 | Lefranc | MNPNSGNT | 73 |
| S0114 | CDRH2 | Lefranc | ISNDGSNE | 77 |
| S0023 | CDRH3 | Lefranc | VKGGGDEYFYYGMDV | 78 |
| S0040 | CDRH3 | Lefranc | TRGGPTLVRGVIIRDAFDF | 79 |
| S0042 | CDRH3 | Lefranc | ATDKRYFDWLLSGFDY | 80 |
| S0057 | CDRH3 | Lefranc | ARDGGDGFDP | 81 |
| S0102 | CDRH3 | Lefranc | ARGGYYGSGSLDY | 82 |
| S0103 | CDRH3 | Lefranc | ARGGPNLVRGVIIRDAFDF | 83 |
| S0114 | CDRH3 | Lefranc | AREGESYPFDY | 84 |
| S0023 | CDRL1 | Lefranc | QGIRND | 85 |
| S0040 | CDRL1 | Lefranc | QSVSNS | 86 |
| S0042 | CDRL1 | Lefranc | QGISSY | 87 |
| S0057 | CDRL1 | Lefranc | QSVSSF | 88 |
| S0102 | CDRL1 | Lefranc | QSLVYSDGNTY | 89 |
| S0103 | CDRL1 | Lefranc | QSVSNS | 86 |
| S0114 | CDRL1 | Lefranc | QSVSSN | 90 |
| S0023 | CDRL2 | Lefranc | SA | 91 |
| S0040 | CDRL2 | Lefranc | GA | 92 |
| S0042 | CDRL2 | Lefranc | AA | 93 |
| S0057 | CDRL2 | Lefranc | DA | 94 |
| S0102 | CDRL2 | Lefranc | KV | 95 |
| S0103 | CDRL2 | Lefranc | GA | 92 |
| S0114 | CDRL2 | Lefranc | GA | 92 |
| S0023 | CDRL3 | Lefranc | LQHNSYPYT | 60 |
| S0040 | CDRL3 | Lefranc | QQYKTWPLT | 61 |
| S0042 | CDRL3 | Lefranc | QRLHSYPRT | 62 |
| S0057 | CDRL3 | Lefranc | QQRSNWIT | 63 |
| S0102 | CDRL3 | Lefranc | MQGTHWPWT | 64 |
| S0103 | CDRL3 | Lefranc | QQYKTWPLT | 61 |
| S0114 | CDRL3 | Lefranc | QQYNNWPYT | 65 |

In some embodiments, anti-siglec antibodies of the present disclosure include variable domains with a set of CDRs, wherein each member of the set includes a CDR amino acid sequence presented herein. In some embodiments, variable domain CDR amino acid sequence sets may include any of those presented in Table 6.

TABLE 6
Variable domain CDR amino acid sequence sets

| | Variable | CDR1 SEQ | CDR2 SEQ | CDR3 SEQ |
|---|---|---|---|---|
| ID# | domain | ID NO | ID NO | ID NO |
| S0023 | VH | 29 | 35 | 41 |
| S0040 | VH | 30 | 36 | 42 |
| S0042 | VH | 31 | 37 | 43 |
| S0057 | VH | 32 | 38 | 44 |
| S0102 | VH | 33 | 39 | 45 |
| S0103 | VH | 30 | 36 | 46 |
| S0114 | VH | 34 | 40 | 47 |
| S0023 | VH | 66 | 72 | 78 |
| S0040 | VH | 67 | 73 | 79 |
| S0042 | VH | 68 | 74 | 80 |
| S0057 | VH | 69 | 75 | 81 |
| S0102 | VH | 70 | 76 | 82 |
| S0103 | VH | 67 | 73 | 83 |
| S0114 | VH | 71 | 77 | 84 |
| S0023 | VL | 48 | 54 | 60 |
| S0040 | VL | 49 | 55 | 61 |
| S0042 | VL | 50 | 56 | 62 |
| S0057 | VL | 51 | 57 | 63 |
| S0102 | VL | 52 | 58 | 64 |
| S0103 | VL | 49 | 55 | 61 |
| S0114 | VL | 53 | 59 | 65 |
| S0023 | VL | 85 | 91 | 60 |
| S0040 | VL | 86 | 92 | 61 |
| S0042 | VL | 87 | 93 | 62 |
| S0057 | VL | 88 | 94 | 63 |
| S0102 | VL | 89 | 95 | 64 |
| S0103 | VL | 86 | 92 | 61 |
| S0114 | VL | 90 | 92 | 65 |

In some embodiments, anti-siglec antibodies of the present disclosure include pairs of variable domain CDR amino acid sequence sets presented herein. In some embodiments, anti-siglec antibodies of the present disclosure include variable domain CDR amino acid sequence set pairs presented in Table 7.

TABLE 7
Variable domain CDR amino acid sequence set pairs

| | CDRH1 | CDRH2 | CDRH3 | CDRL1 | CDRL2 | CDRL3 |
|---|---|---|---|---|---|---|
| | SEQ | SEQ | SEQ | SEQ | SEQ | SEQ |
| ID# | ID NO | ID NO | ID NO | ID NO | ID NO | ID NO |
| S0023 | 29 | 35 | 41 | 48 | 54 | 60 |
| S0040 | 30 | 36 | 42 | 49 | 55 | 61 |
| S0042 | 31 | 37 | 43 | 50 | 56 | 62 |
| S0057 | 32 | 38 | 44 | 51 | 57 | 63 |
| S0102 | 33 | 39 | 45 | 52 | 58 | 64 |
| S0103 | 30 | 36 | 46 | 49 | 55 | 61 |
| S0114 | 34 | 40 | 47 | 53 | 59 | 65 |
| S0023 | 66 | 72 | 78 | 85 | 91 | 60 |
| S0040 | 67 | 73 | 79 | 86 | 92 | 61 |
| S0042 | 68 | 74 | 80 | 87 | 93 | 62 |
| S0057 | 69 | 75 | 81 | 88 | 94 | 63 |
| S0102 | 70 | 76 | 82 | 89 | 95 | 64 |
| S0103 | 67 | 73 | 83 | 86 | 92 | 61 |
| S0114 | 71 | 77 | 84 | 90 | 92 | 65 |

In some embodiments, anti-siglec antibodies of the present disclosure include one or more framework regions (FRs). FRs may include amino acid sequences derived from variable domain amino acid sequences provided in Table 2. FRs may be encoded by nucleic acid sequences derived from one or more variable domain nucleic acid sequences provided in Table 3. In some embodiments, anti-siglec antibody FRs may include amino acid sequences according to any of those presented in Table 8, or fragments thereof. In some embodiments, anti-siglec antibodies of the present disclosure include FRs that include amino acid sequence variants of those listed. Amino acid fragments or variants included in anti-siglec antibody FRs may include from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the amino acid sequences listed.

TABLE 8
FR amino acid sequences

| | | Numbering | | SEQ ID |
|---|---|---|---|---|
| ID# | Region | Scheme | Sequence | NO |
| S0023 | FRH1 | Kabat | EVQLLESGGGLVQPGGSLRLSCAASGFTFS | 96 |
| S0040 | FRH1 | Kabat | QVQLVQSGAEVKKPGASVKVSCKASGYTFT | 97 |
| S0042 | FRH1 | Kabat | QVQLVQSGTEVKKPGASVKVSCKVSGYTLS | 98 |
| S0057 | FRH1 | Kabat | QGQLVQSGAEVKKPGASVKVSCKASGYTFN | 99 |
| S0102 | FRH1 | Kabat | QVQLVESGGGVVQPGRSLRLSCAASGFTFS | 100 |
| S0103 | FRH1 | Kabat | QVQLVQSGAEVKKPGASVKVSCKASGYTFT | 97 |
| S0114 | FRH1 | Kabat | QVQLVESGGGVVQPGRSLRLSCAASGFTFS | 100 |
| S0023 | FRH2 | Kabat | WVRQAPGKGLEWVS | 101 |
| S0040 | FRH2 | Kabat | WVRQATGQGLEWMG | 102 |
| S0042 | FRH2 | Kabat | WVRQAPGKGLEWMG | 103 |
| S0057 | FRH2 | Kabat | WVRQAPGQGLEWMG | 104 |
| S0102 | FRH2 | Kabat | WVRQAPGKGLEWVA | 105 |
| S0103 | FRH2 | Kabat | WVRQATGQGLEWMG | 102 |
| S0114 | FRH2 | Kabat | WVRQAPGKGLEWVA | 105 |
| S0023 | FRH3 | Kabat | RFTISRDNSKNMMSLQMNSLRAVDTAVYYCVK | 106 |
| S0040 | FRH3 | Kabat | RVTMTRNTSINTAYMELSSLRSEDTAVYYCTR | 107 |
| S0042 | FRH3 | Kabat | RVIMTEDTSTDTAYMKLSSLRSEDTAVYYCAT | 108 |
| S0057 | FRH3 | Kabat | RVTLTTDTSTSTAHMELRSLRSDDTAAYYCAR | 109 |
| S0102 | FRH3 | Kabat | RFTISRDNSKNTLYLQMNSLRAEDTAVYYCAR | 110 |
| S0103 | FRH3 | Kabat | RVTMTRDTSVNTAYMELSSLRSEDTAVYYCAR | 111 |
| S0114 | FRH3 | Kabat | RFTISRDYSKNTLYLQMNSLRAXDTAVYYCAR | 112 |
| S0023 | FRH4 | Kabat | WGQGTTVTVSS | 113 |
| S0040 | FRH4 | Kabat | WGQGTMVTVSS | 114 |
| S0042 | FRH4 | Kabat | WGQGTLVTVSS | 115 |
| S0057 | FRH4 | Kabat | WGQGTLVTVSS | 115 |
| S0102 | FRH4 | Kabat | WGQGTLVTVSS | 115 |
| S0103 | FRH4 | Kabat | WGQGTMVTVSS | 114 |
| S0114 | FRH4 | Kabat | WGQGTLVTVSS | 115 |
| S0023 | FRL1 | Kabat | DIQMTQSPSSLSASVGDRVTITC | 116 |
| S0040 | FRL1 | Kabat | EIVMTQSPATLSVSPGERATLSC | 117 |
| S0042 | FRL1 | Kabat | DIQLTQSPSFLSASVGDRVTITC | 118 |
| S0057 | FRL1 | Kabat | EIVLTQSPATLSLSPGERATLSC | 119 |
| S0102 | FRL1 | Kabat | DVVMTQSPLSLPVTLGQPASISC | 120 |
| S0103 | FRL1 | Kabat | EIVMTQSPATLSVSPGERATLSC | 117 |
| S0114 | FRL1 | Kabat | EIVMTQSPATLSVSPGERATLSC | 117 |
| S0023 | FRL2 | Kabat | WYQQKPGKAPKRLIY | 121 |
| S0040 | FRL2 | Kabat | WYQQKPGQAPRLLIY | 122 |
| S0042 | FRL2 | Kabat | WYQQKPGKAPKLLIF | 123 |
| S0057 | FRL2 | Kabat | WYQQKPGQAPRLLIF | 124 |
| S0102 | FRL2 | Kabat | WFQQRPGQSPRRLIY | 125 |
| S0103 | FRL2 | Kabat | WYQQKPGQAPRLLIY | 122 |
| S0114 | FRL2 | Kabat | WYQQKPGQAPRLLIY | 122 |
| S0023 | FRL3 | Kabat | GVPSRFSGSGSGTEFTLTISSLQPEDFATYYC | 126 |
| S0040 | FRL3 | Kabat | GIPARFSGSGSGTEFTLTISSLQSEDFAVYYC | 127 |
| S0042 | FRL3 | Kabat | GVPSRFSGSGSGTEFTLTISSLQPEDFATYYC | 126 |
| S0057 | FRL3 | Kabat | GIPARFSGSGSGTDFTLTISSLEPEDFAVYYC | 128 |
| S0102 | FRL3 | Kabat | GVPDRFSGSGSGTDFTLKISRVEAEDVGVYYC | 129 |
| S0103 | FRL3 | Kabat | GIPARFSGSGSGIEFTLTISSLQSEDFAVYYC | 130 |
| S0114 | FRL3 | Kabat | GIPARFSGSGSGTEFTLTISSLQSEDFAVYYC | 127 |
| S0023 | FRL4 | Kabat | FGQGTPLDIKRA | 131 |
| S0040 | FRL4 | Kabat | FGGGTKVEIKRA | 132 |
| S0042 | FRL4 | Kabat | FGQGTKVEIKRA | 133 |
| S0057 | FRL4 | Kabat | FGQGTRLEIKRA | 134 |
| S0102 | FRL4 | Kabat | FGQGTKVEIKRA | 133 |
| S0103 | FRL4 | Kabat | FGGGTKVEIKRA | 132 |
| S0114 | FRL4 | Kabat | FGQGTKLEIKRA | 135 |
| S0023 | FRH1 | Lefranc | EVQLLESGGGLVQPGGSLRLSCAAS | 136 |
| S0040 | FRH1 | Lefranc | QVQLVQSGAEVKKPGASVKVSCKAS | 137 |
| S0042 | FRH1 | Lefranc | QVQLVQSGTEVKKPGASVKVSCKVS | 138 |
| S0057 | FRH1 | Lefranc | QGQLVQSGAEVKKPGASVKVSCKAS | 139 |
| S0102 | FRH1 | Lefranc | QVQLVESGGGVVQPGRSLRLSCAAS | 140 |
| S0103 | FRH1 | Lefranc | QVQLVQSGAEVKKPGASVKVSCKAS | 137 |
| S0114 | FRH1 | Lefranc | QVQLVESGGGVVQPGRSLRLSCAAS | 140 |
| S0023 | FRH2 | Lefranc | MSWVRQAPGKGLEWVST | 141 |
| S0040 | FRH2 | Lefranc | INWVRQATGQGLEWMGW | 142 |
| S0042 | FRH2 | Lefranc | IHWVRQAPGKGLEWMGG | 143 |
| S0057 | FRH2 | Lefranc | ISWVRQAPGQGLEWMGW | 144 |
| S0102 | FRH2 | Lefranc | MHWVRQAPGKGLEWVAV | 145 |
| S0103 | FRH2 | Lefranc | INWVRQATGQGLEWMGW | 142 |
| S0114 | FRH2 | Lefranc | MHWVRQAPGKGLEWVAV | 145 |
| S0023 | FRH3 | Lefranc | YYADSVKGRFTISRDNSKNMMSLQMNSLRAVDTAVYYC | 146 |
| S0040 | FRH3 | Lefranc | GYAQNFQGRVTMTRNTSINTAYMELSSLRSEDTAVYYC | 147 |
| S0042 | FRH3 | Lefranc | IYAQKFQGRVIMTEDTSTDTAYMKLSSLRSEDTAVYYC | 148 |
| S0057 | FRH3 | Lefranc | RSTQKFQGRVTLTTDTSTSTAHMELRSLRSDDTAAYYC | 149 |
| S0102 | FRH3 | Lefranc | YYTDSVKGRFTISRDNSKNTLYLQMNSLRAEDTAVYYC | 150 |
| S0103 | FRH3 | Lefranc | GYAQNFQGRVTMTRDTSVNTAYMELSSLRSEDTAVYYC | 151 |
| S0114 | FRH3 | Lefranc | YYADSVKGRFTISRDYSKNTLYLQMNSLRAXDTAVYYC | 152 |
| S0023 | FRH4 | Lefranc | WGQGTTVTVSS | 113 |
| S0040 | FRH4 | Lefranc | WGQGTMVTVSS | 114 |
| S0042 | FRH4 | Lefranc | WGQGTLVTVSS | 115 |
| S0057 | FRH4 | Lefranc | WGQGTLVTVSS | 115 |
| S0102 | FRH4 | Lefranc | WGQGTLVTVSS | 115 |
| S0103 | FRH4 | Lefranc | WGQGTMVTVSS | 114 |
| S0114 | FRH4 | Lefranc | WGQGTLVTVSS | 115 |
| S0023 | FRL1 | Lefranc | DIQMTQSPSSLSASVGDRVTITCRAS | 153 |
| S0040 | FRL1 | Lefranc | EIVMTQSPATLSVSPGERATLSCRAS | 154 |
| S0042 | FRL1 | Lefranc | DIQLTQSPSFLSASVGDRVTITCRAS | 155 |
| S0057 | FRL1 | Lefranc | EIVLTQSPATLSLSPGERATLSCRAS | 156 |
| S0102 | FRL1 | Lefranc | DVVMTQSPLSLPVTLGQPASISCRSS | 157 |
| S0103 | FRL1 | Lefranc | EIVMTQSPATLSVSPGERATLSCRAS | 154 |
| S0114 | FRL1 | Lefranc | EIVMTQSPATLSVSPGERATLSCRAS | 154 |
| S0023 | FRL2 | Lefranc | LGWYQQKPGKAPKRLIY | 158 |
| S0040 | FRL2 | Lefranc | FAWYQQKPGQAPRLLIY | 159 |
| S0042 | FRL2 | Lefranc | LAWYQQKPGKAPKLLIF | 160 |
| S0057 | FRL2 | Lefranc | LAWYQQKPGQAPRLLIF | 161 |
| S0102 | FRL2 | Lefranc | LNWFQQRPGQSPRRLIY | 162 |
| S0103 | FRL2 | Lefranc | FAWYQQKPGQAPRLLIY | 159 |
| S0114 | FRL2 | Lefranc | LAWYQQKPGQAPRLLIY | 163 |
| S0023 | FRL3 | Lefranc | SSLQSGVPSRFSGSGSGTEFTLTISSLQPEDFATYYC | 164 |
| S0040 | FRL3 | Lefranc | FTRATGIPARFSGSGSGTEFTLTISSLQSEDFAVYYC | 165 |
| S0042 | FRL3 | Lefranc | STLQSGVPSRFSGSGSGTEFTLTISSLQPEDFATYYC | 166 |
| S0057 | FRL3 | Lefranc | SNRATGIPARFSGSGSGTDFTLTISSLEPEDFAVYYC | 167 |
| S0102 | FRL3 | Lefranc | SNRDSGVPDRFSGSGSGTDFTLKISRVEAEDVGVYYC | 168 |
| S0103 | FRL3 | Lefranc | FTRATGIPARFSGSGSGIEFTLTISSLQSEDFAVYYC | 169 |
| S0114 | FRL3 | Lefranc | FFRATGIPARFSGSGSGTEFTLTISSLQSEDFAVYYC | 170 |
| S0023 | FRL4 | Lefranc | FGQGTPLDIKRA | 131 |
| S0040 | FRL4 | Lefranc | FGGGTKVEIKRA | 132 |
| S0042 | FRL4 | Lefranc | FGQGTKVEIKRA | 133 |
| S0057 | FRL4 | Lefranc | FGQGTRLEIKRA | 134 |
| S0102 | FRL4 | Lefranc | FGQGTKVEIKRA | 133 |
| S0103 | FRL4 | Lefranc | FGGGTKVEIKRA | 132 |
| S0114 | FRL4 | Lefranc | FGQGTKLEIKRA | 135 |

Anti-siglec antibodies according to the present disclosure may be prepared using any of the antibody sequences (e.g., variable domain amino acid sequences, variable domain amino acid sequence pairs, CDR amino acid sequences, variable domain CDR amino acid sequence sets, variable domain CDR amino acid sequence set pairs, and/or framework region amino acid sequences) presented herein, any may be prepared, for example, as monoclonal antibodies, multispecific antibodies, chimeric antibodies, antibody mimetics, scFvs, or antibody fragments. In some embodiments, anti-siglec antibodies using any of the antibody sequences presented herein may be prepared as IgA, IgD, IgE, IgG, or IgM antibodies. When prepared as mouse IgG antibodies, anti-siglec antibodies may be prepared as IgG1, IgG2a, IgG2b, or IgG3 isotypes. When prepared as human IgG antibodies, anti-siglec antibodies may be prepared as IgG1, IgG2, IgG3, or IgG4 isotypes. Anti-siglec antibodies prepared as human or humanized antibodies may include one or more human constant domains.

### Siglec Protein Antigens

In some embodiments, anti-siglec antibodies bind to siglec12 protein antigens. In some embodiments siglec12 protein antigens include any of those listed in Table 9. Some siglec12 proteins antigens may be post-translationally processed or recombinantly expressed to exclude leader sequences (underlined in the Table). In some embodiments, siglec12 proteins may include variants or fragments of the sequences listed.

TABLE 9
Siglec12 protein antigen sequences

| | | SEQ |
|---|---|---|
| Antigen | Sequence | ID NO |
| siglec12 | MLLLLLLLPPLLCGRVGAKEQKDYLLTMQKSVTVQEGLCVS | 171 |
| extracellular | VLCSFSYPQNGWTASDPVHGYWFRAGDHVSRNIPVATNNPA | |
| domain | RAVQEETRDRFHLLGDPQNKDCTLSIRDTRESDAGTYVFCVE | |
| | RGNMKWNYKYDQLSVNVTASQDLLSRYRLEVPESVTVQEG | |
| | LCVSVPCSVLYPHYNWTASSPVYGSWFKEGADIPWDIPVATN | |
| | TPSGKVQEDTHGRFLLLGDPQTNNCSLSIRDARKGDSGKYYF | |
| | QVERGSRKWNYIYDKLSVHVTALTHMPTFSIPGTLESGHPRN | |
| | LTCSVPWACEQGTPPTITWMGASVSSLDPTITRSSMLSLIPQPQ | |
| | DHGTSLTCQVTLPGAGVTMTRAVRLNISYPPQNLTMTVFQG | |
| | DGTASTTLRNGSALSVLEGQSLHLVCAVDSNPPARLSWTWG | |
| | SLTLSPSQSSNLGVLELPRVHVKDEGEFTCRAQNPLGSQHISL | |
| | SLSLQNEYTGKMRPISGVTLGA | |
| siglec12 long | MLLLLLLLPPLLCGRVGAKEQKDYLLTMQKSVTVQEGLCVS | 172 |
| form | VLCSFSYPQNGWTASDPVHGYWFRAGDHVSRNIPVATNNPA | |
| | RAVQEETRDRFHLLGDPQNKDCTLSIRDTRESDAGTYVFCVE | |
| | RGNMKWNYKYDQLSVNVTASQDLLSRYRLEVPESVTVQEG | |
| | LCVSVPCSVLYPHYNWTASSPVYGSWFKEGADIPWDIPVATN | |
| | TPSGKVQEDTHGRFLLLGDPQTNNCSLSIRDARKGDSGKYYF | |
| | QVERGSRKWNYIYDKLSVHVTALTHMPTFSIPGTLESGHPRN | |
| | LTCSVPWACEQGTPPTITWMGASVSSLDPTITRSSMLSLIPQPQ | |
| | DHGTSLTCQVTLPGAGVTMTRAVRLNISYPPQNLTMTVFQG | |
| | DGTASTTLRNGSALSVLEGQSLHLVCAVDSNPPARLSWTWG | |
| | SLTLSPSQSSNLGVLELPRVHVKDEGEFTCRAQNPLGSQHISL | |
| | SLSLQNEYTGKMRPISGVTLGAFGGAGATALVFLYFCIIFVVV | |
| | RSCRKKSARPAVGVGDTGMEDANAVRGSASQGPLIESPADD | |
| | SPPHHAPPALATPSPEEGEIQYASLSFHKARPQYPQEQEAIGYE | |
| | YSEINIPK | |
| siglec12 | MLLPLLWANEERDSGGWADPRFSTASQDLLSRYRLEVPESVT | 173 |
| shorter | VQEGLCVSVPCSVLYPHYNWTASSPVYGSWFKEGADIPWDIP | |
| isoform | VATNTPSGKVQEDTHGRFLLLGDPQTNNCSLSIRDARKGDSG | |
| | KYYFQVERGSRKWNYIYDKLSVHVTALTHMPTFSIPGTLESG | |
| | HPRNLTCSVPWACEQGTPPTITWMGASVSSLDPTITRSSMLSL | |
| | IPQPQDHGTSLTCQVTLPGAGVTMTRAVRLNISYPPQNLTMT | |
| | VFQGDGTASTTLRNGSALSVLEGQSLHLVCAVDSNPPARLSW | |
| | TWGSLTLSPSQSSNLGVLELPRVHVKDEGEFTCRAQNPLGSQ | |
| | HISLSLSLQNEYTGKMRPISGVTLGAFGGAGATALVFLYFCIIF | |
| | VVVRSCRKKSARPAVGVGDTGMEDANAVRGSASQGPLIESP | |
| | ADDSPPHHAPPALATPSPEEGEIQYASLSFHKARPQYPQEQEA | |
| | IGYEYSEINIPK | |
| S12-V1 | MLLLLLLLPPLLCGRVGAKEQKDYLLTMQKSVTVQEGLCVS | 174 |
| | VLCSFSYPQNGWTASDPVHGYWFRAGDHVSRNIPVATNNPA | |
| | RAVQEETRDRFHLLGDPQNKDCTLSIRDTRESDAGTYVFCVE | |
| | RGNMKWNYKYDQLSVNVT | |
| S12-V2C1 | MLLLLLLLPPLLCGRVGAKEASQDLLSRYRLEVPESVTVQEG | 175 |
| | LCVSVPCSVLYPHYNWTASSPVYGSWFKEGADIPWDIPVATN | |
| | TPSGKVQEDTHGRFLLLGDPQTNNCSLSIRDARKGDSGKYYF | |
| | QVERGSRKWNYIYDKLSVHVTALTHMPTFSIPGTLESGHPRN | |
| | LTCSVPWACEQGTPPTITWMGASVSSLDPTITRSSMLSLIPQPQ | |
| | DHGTSLTCQVTLPGAGVTMTRAVR | |
| S12-C2 | MLLLLLLLPPLLCGRVGAKEPQNLTMTVFQGDGTASTTLRNG | 176 |
| | SALSVLEGQSLHLVCAVDSNPPARLSWTWGSLTLSPSQSSNL | |
| | GVLELPRVHVKDEGEFTCRAQNPLGSQHISLSLSLQNEYTGK | |
| | MRPISGVTLGA | |

In some embodiments, anti-siglec antibodies of the present disclosure bind to siglec12 protein epitopes on siglec12 protein antigens described herein. Such siglec12 protein epitopes may include or be included within a siglec12 protein antigen amino acid sequence listed in Table 9. In some embodiments, anti-siglec antibodies of the present disclosure bind to siglec12 protein epitopes that include a region formed by a complex of a siglec12 protein epitope with another protein or epitope.

In some embodiments, siglec12 protein antigens may be prepared as fusion proteins. Siglec12 protein antigens may be prepared as fusion proteins with antibody Fc regions. Fusions with antibody Fc regions may be prepared to facilitate immunization and generation of siglec12 protein antigen-specific immune response. In some embodiments, siglect12 protein antigens are prepared as fusion proteins with mouse Fc regions. Such Fc regions may include Fc2a fragment (UniProtKB PO1863.1, amino acids 98-330, PRGPTIKPCPPCKCPAPNLLGGPSVFIFPPKIKDVLMISLSPIVTCVVVDVSEDDPDVQISW FVNNVEVHTAQTQTHREDYNSTLRVVSALPIQHQDWMSGKEFKCKVNNKDLPAPIERTI SKPKGSVRAPQVYVLPPPEEEMTKKQVTLTCMVTDFMPEDIYVEWTNNGKTELNYKNT EPVLDSDGSYFMYSKLRVEKKNWVERNSYSCSVVHEGLHNHHTTKSFSRTPGK, SEQ ID NO: 177) or may include mouse 2cFc (EPRVPITQNPCPPLKECPPCAAPDLLGGPSVFIFPPKIKDVLMISLSPMVTCVVVDVSEDD PDVQISWFVNNVEVHTAQTQTHREDYNSTLRVVSALPIQHQDWMSGKEFKCKVNNRA LPSPIEKTISKPRGPVRAPQVYVLPPPAEEMTKKEFSLTCMITGFLPAEIAVDWTSNGRTE QNYKNTATVLDSDGSYFMYSKLRVQKSTWERGSLFACSVVHEGLHNHLTTKTISRSLG K, SEQ ID NO: 178). Mouse Fc regions may be used in place of human Fc when carrying out immunizations in mice to minimize generation of antibodies against Fc regions.

### II. Methods

In some embodiments, the present disclosure provides methods of using and evaluating anti-siglec antibodies for therapeutic and diagnostic applications.

### Therapeutic Applications

In some embodiments, the present disclosure provides methods of treating therapeutic indications using anti-siglec antibodies (e.g., any of those described herein). The term “therapeutic indication,” as used herein, refers to any disease, symptom, condition, or disorder that may be alleviated, stabilized, improved, cured, or otherwise addressed by some form of treatment or other therapeutic intervention. In some embodiments, the present disclosure provides methods of treating therapeutic indications in subjects by providing antibodies disclosed herein.

As used herein the terms “treat,” “treatment,” and the like, refer to any actions taken to offer relief from or alleviation of pathological processes. As it relates to any of the therapeutic indications recited herein, the terms “treat,” “treatment,” and the like mean to relieve or alleviate at least one symptom associated with such indications, or to slow or reverse the progression or anticipated progression of such indications.

In some embodiments, the present disclosure provides methods of treating therapeutic indications in subjects using anti-siglec antibodies (e.g., any of the antibodies described herein). Such antibodies may include human variable domains. Therapeutic indications may include cancer-related indications, e.g., leukemia, e.g., AML and/or CML. The methods of treatment may include administering anti-siglec antibodies to subjects, e.g., by injection or infusion.

### Cancer-Related Indications

In some embodiments, therapeutic indications include cancer-related indications. The term “cancer” refers to a collection of diseases characterized by dysfunctional cell growth and division, in some cases spreading between bodily regions. As used herein, the term “cancer-related indication” refers to any disease, disorder, or condition pertaining to cancer, cancer treatment, or pre-cancerous conditions. Cancer-related indications include, but are not limited to, pathological conditions characterized by malignant neoplastic growths, tumors, and/or hematological malignancies. In some embodiments, methods of the present disclosure include treatment of cancer-related indications with anti-siglec antibodies (e.g., any of those described herein).

Cancer-related indications include leukemias, which are cancers of the blood or blood forming tissues (e.g., bone marrow and lymphatic system). Leukemias are typically classified by progression and affected cell type. Acute leukemias develop quickly while chronic leukemias develop slowly with worsening effects over time. Lymphocytic leukemias affect lymphocytes, typically due to mutations in cells that make lymphocytes. Myelogenous leukemias involve myeloid cells responsible for blood cell production.

In some embodiments, methods of the present disclosure include treatment of acute myeloid leukemia (AML) and/or chronic myeloid leukemia (CML) with anti-siglec antibodies (e.g., any of those described herein). AML involves rapid abnormal myeloblast accumulation, typically in the bone marrow. CML involves mutation in immature myeloid cells leading to a more gradual accumulation of leukemia cells over time that can spread to other parts of the body.

Additional cancer-related indications include, but are not limited to, any of those listed in Table 10 below. Anti-siglec antibodies used according to cancer-related indication treatment methods described herein may bind to siglec12 associated with cancer cells, including cancer cells associated with any of the cancer-related indications listed in Table 10.

TABLE 10
Cancer-related indications
Cancer-related indications

Cervical squamous cell carcinoma and endocervical adenocarcinoma
Head and neck squamous cell carcinoma
Kidney renal clear cell carcinoma
Esophageal carcinoma
Stomach and esophageal cancer
Glioblastoma multiforme
Kidney PAN cancer
Sarcoma
Kidney chromophobe
Kidney renal papillary cell carcinoma
Thyroid carcinoma
Uterine corpus endometrial carcinoma

Anti-siglec antibodies used according to cancer-related indication treatment methods described herein may bind to siglec12 associated with solid epithelial tumors. Such tumors have been shown by immunohistochemical analysis to express siglec12 (United States Publication Number US2020/0003779). According to some methods, siglec12-associated cancer cells targeted according to treatment methods of the present disclosure include hematopoietic cells and/or lymphoid cells.

In some embodiments, methods of the present disclosure include treatment of AML and/or CML in subjects by providing anti-siglec antibodies (e.g., any of the anti-siglec antibodies described herein). Siglec-3 or CD33 is expressed on AML cells, including AML blasts in AML patients. Gemtuzumab Ozogamicin (GO, MYLOTARG®, Pfizer, New York, NY) is an antibody drug conjugate that targets CD33 and includes a calicheamicin warhead. GO is the only currently listed form of immunotherapy for AML. In some embodiments, anti-siglec antibodies of the present disclosure target siglec12 on AML and/or CML cells in subjects treated according to methods described herein.

In some embodiments, the present disclosure provides methods of treating subjects with cancer by administering an anti-siglec12 antibodies (e.g., any of those described herein). The anti-siglec12 antibodies may include VH amino acid sequences according to any of those presented in Table 2 and/or may be encoded by any of the nucleic acid sequences presented in Table 3. The anti-siglec12 antibodies may include VL amino acid sequences according to any of those presented in Table 2 and/or may be encoded by any of the nucleic acid sequences presented in Table 3. The anti-siglec12 antibodies may bind subject cancer cells. Such cancer cells may include AML cancer cells. In some embodiments, the anti-siglec12 antibodies may be internalized by bound cells. Such antibodies may be ADCs. Therapeutic agents associated with such ADCs may include cytotoxins. Such toxins may include, but are not limited to, radioisotopes, saporins, taxanes, vinca alkaloids, anthracyclines, calicheamicins, duocarmycins, pyrrolobenzodiazepine dimers, and platinum-based agents. Calicheamicins may include MYLOTARG®. In some embodiments, the anti-siglec12 antibodies include a VH with the amino acid sequence of SEQ ID NO: 4 and a VL with the amino acid sequence of SEQ ID NO: 11. The anti-siglec12 antibodies may be whole antibodies or may be antibody fragments (e.g., Fab fragments). The anti-siglec12 antibodies may have an equilibrium dissociation constant (Kd) for binding to siglec12 of from about 0.001 nM to about 10,000 nM (e.g., from about 0.001 nM to about 0.1 nM, from about 0.01 nM to about 1 nM, from about 0.5 nM to about 5 nM, from about 1 nM to about 10 nM, from about 2 nM to about 20 nM, from about 5 nM to about 50 nM, from about 15 nM to about 100 nM, from about 25 nM to about 250 nM, from about 100 nM to about 1,000 nM, or from about 500 nM to about 10,000 nM).

### Diagnostic Applications

In some embodiments, the present disclosure provides diagnostic methods involving use of anti-siglec antibodies. Such methods may include detecting siglecs using anti-siglec antibodies (e.g., any of the antibodies described herein). Such methods may include contacting subjects or subject samples with anti-siglec antibodies (e.g., any of those described herein). The anti-siglec antibodies may bind siglec12. The anti-siglec antibodies may include human variable domains. Antibodies used for detection methods may include a detectable label. Detection methods may include the use of detection reagents to detect bound antibodies. As used herein, the term “detection reagent” refers to any compound or substance used to visualize or otherwise observe an object (e.g., a bound antibody or detectable label) or event. Detection reagents may include secondary antibodies or other high affinity compounds (e.g., biotin or avidin) that bind to antibodies being detected or associated conjugates. Detection reagents may be or include substrates for detection of enzymatic detectable labels (e.g., associated with a primary or secondary antibody).

Diagnostic applications of the present disclosure may include detecting siglecs in subject samples that include cells. In some embodiment cell-associated siglecs may be detected. Cell-associated siglecs may be detected in subject samples by fluorescence-associated cell sorting (FACS) analysis. In some embodiments, siglecs may be detected in subject samples by immunohistochemistry. Such methods may include the use of colorimetric-based systems or immunofluorescence-based systems for siglec detection.

In some embodiments, the present disclosure provides methods of stratifying subjects based on detection of siglecs in subjects or subject samples. Such methods may include detecting siglecs in subjects or subject samples according to any of the methods described herein (e.g., using anti-siglec antibodies) and classifying subjects according to type and/or level of siglecs detected. In some embodiments, subjects may be classified according to the presence or absence of siglec12 and/or level of siglec12 in subjects or subject samples. Subjects may be further classified according to the presence or absence of specific siglec12 extracellular subdomains and/or levels of specific siglec 12 extracellular subdomains in subjects or subject samples. Classifications used in subject stratification may include, but are not limited to, classifications by disease type, disease prognosis or severity, suitability for treatment, and type of treatment most likely to be successful or appropriate.

### III. Definitions

Associated with: As used herein, the terms “associated with,” “conjugated,” “linked,” “attached,” and “tethered,” when used with respect to two or more entities, means that the entities are physically associated or connected with one another, either directly or via one or more moieties that serve as linking agents, to form a structure that is sufficiently stable so that the entities remain physically associated, e.g., under working conditions, e.g., under physiological conditions. An “association” need not be through covalent chemical bonding and may include other forms of association or bonding sufficiently stable such that the “associated” entities remain physically associated, e.g., ionic or hydrogen bonding or a hybridization based connectivity.

Epitope: As used herein, an “epitope” refers to a surface or region on one or more entities that is capable of interacting with an antibody or other binding biomolecule. For example, a protein epitope may contain one or more amino acids and/or post-translational modifications (e.g., phosphorylated residues) which interact with an antibody. In some embodiments, an epitope may be a “conformational epitope,” which refers to an epitope involving a specific three-dimensional arrangement of the entity(ies) having or forming the epitope. For example, conformational epitopes of proteins may include combinations of amino acids and/or post-translational modifications from folded, non-linear stretches of amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Sample: As used herein, the term “sample” or “biological sample” refers to a subset of its tissues, cells or component parts (e.g. body fluids, including but not limited to blood, serum, plasma, mucus, lymphatic fluid, synovial fluid, cerebrospinal fluid, saliva, amniotic fluid, and urine). Samples may further include a homogenate, lysate, or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, and organs. Samples may further refer to a medium, such as a nutrient broth or gel, which may contain cellular components or other biological materials, such as proteins (e.g., antibodies) or nucleic acid molecules.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. A subject receiving, requiring, eligible for, or seeking medical treatment is referred to herein as a “patient.”

Target: As used herein, the term “target” refers to an entity of interest or attention, which may include a subject, an organ, a tissue, a cell, a protein, a nucleic acid, biomolecule, or a group, complex, or portion of any of the foregoing. In some embodiments, a target may be a protein or epitope thereof for which an antibody has affinity or for which an antibody is desired, designed, or developed to have affinity for. As used herein, the term “target” may also be used to refer to an activity of an agent that is directed to a particular object. For example, an antibody that has affinity for a specific protein “X” may be said to target protein X or may be referred to as an antibody targeting protein X or referred to as a protein X-targeting antibody. Similarly, an object that is the subject of an agent's activity may be referred to as a “targeted” object. For example, where an antibody has affinity for a specific protein “X,” protein X may be referred to as being targeted by the antibody.

### IV. Equivalents and Scope

While various embodiments of the invention have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The invention includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present

disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. Siglec12 is a Novel Target for Acute Myeloid Leukemia

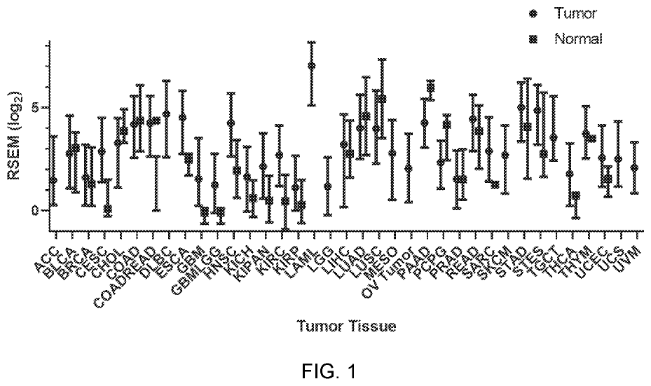

Siglec12 was reported to be detected in solid epithelial tumors using immunohistochemistry (United States Publication Number US 2020/0003779). However, immunohistochemistry is a non-quantitative methodology and understanding relative siglec12 expression levels between tumor and normal tissue is critical to understanding which cancers will be treatable using a siglec12-targeted immunotherapeutic approach. mRNA levels of a specific target in tissue is an accepted measure of relative expression levels of a specific target. RNAseq data (mRNA levels quantified via next gen sequencing, NGS) from various public databases (TCGA project, Depmap Portal, CCLE, Blueprint Project, and GTEx) were analyzed to discover which cancers make the most suitable targets for siglec12-targeted immunotherapy. Results presented in FIG. 1 show differential expression levels of siglec12 between normal tissue and tumors originating in the same tissue (based in whole or in part on data generated by the TCGA Research Network). These results are summarized in Table 11 and reveal that certain solid tumors are preferable cancer targets for siglec12-targeted immunotherapy than others, namely: cervical squamous cell carcinoma and endocervical adenocarcinoma; head and neck squamous cell carcinoma; kidney renal clear cell carcinoma; esophageal carcinoma and stomach and esophageal cancer.

TABLE 11
Increased siglec12 mRNA expression in different solid tumors

| | Siglec12 expression |
|---|---|
| Tumor type | (fold increase) |
| Cervical squamous cell carcinoma | 7 |
| and endocervical adenocarcinoma | |
| Head and neck squamous cell carcinoma | 5 |
| Kidney renal clear cell carcinoma | 5 |
| Esophageal carcinoma | 4 |
| Stomach and esophageal cancer | 4 |
| Glioblastoma multiforme | 3 |
| Kidney PAN cancer | 3 |
| Sarcoma | 3 |
| Kidney chromophobe | 2 |
| Kidney renal papillary cell carcinoma | 2 |
| Thyroid carcinoma | 2 |
| Uterine corpus endometrial carcinoma | 2 |

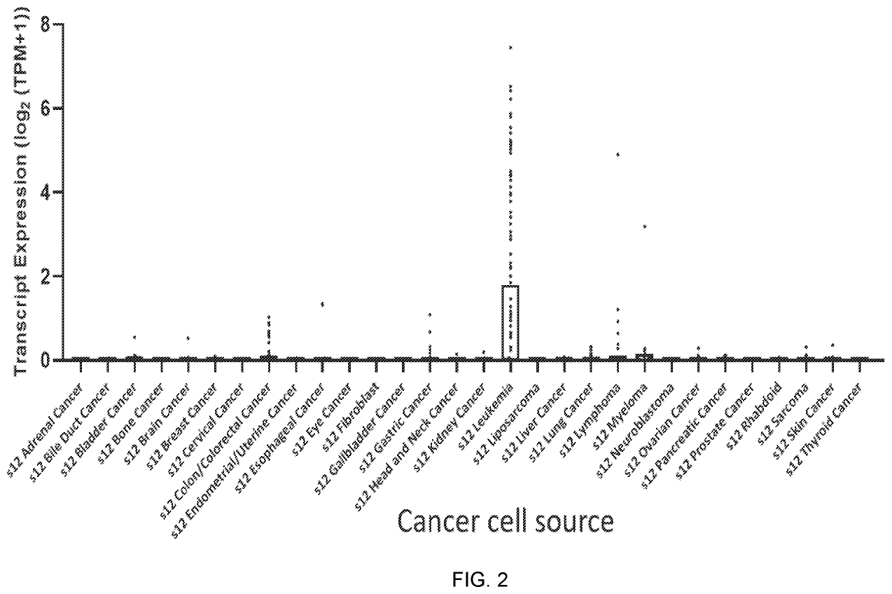

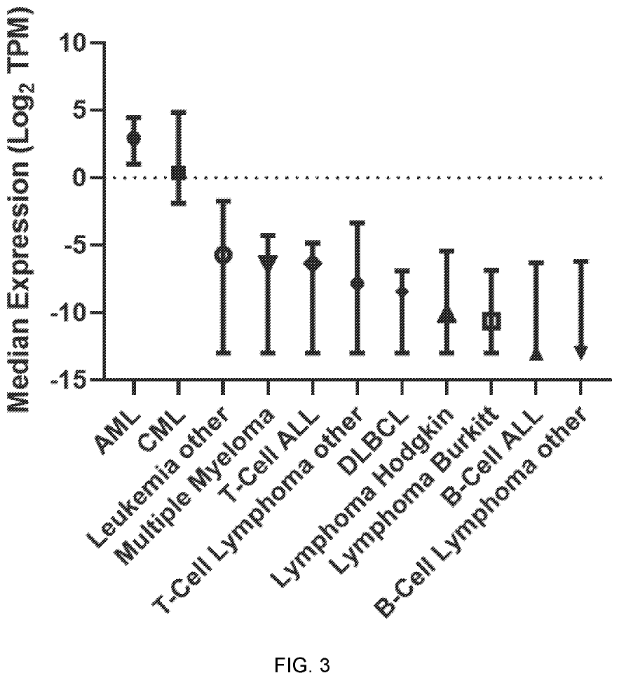

Further database analysis [DepMap Portal, Broad (2020): DepMap 20Q2 Public, v4] revealed that when measuring mRNA levels (RNAseq) across 1304 different tumor cell lines, siglec12 was most highly expressed in the leukemia cell lines, as shown in FIG. 2. In cancer cell lines of hematopoietic and lymphoid tissue origin, both AML (acute myeloid leukemia) and CML (chronic myeloid leukemia) cell lines had significantly greater siglec12 mRNA expression than the other cell lines [based on 2020 Broad Institute CCLE (Cancer Cell Line Encyclopedia) mRNA expression (RNAseq) data, see FIG. 3]. Between AML and CML, median siglec12 mRNA expression in AML was 6-fold greater than median levels in CML.

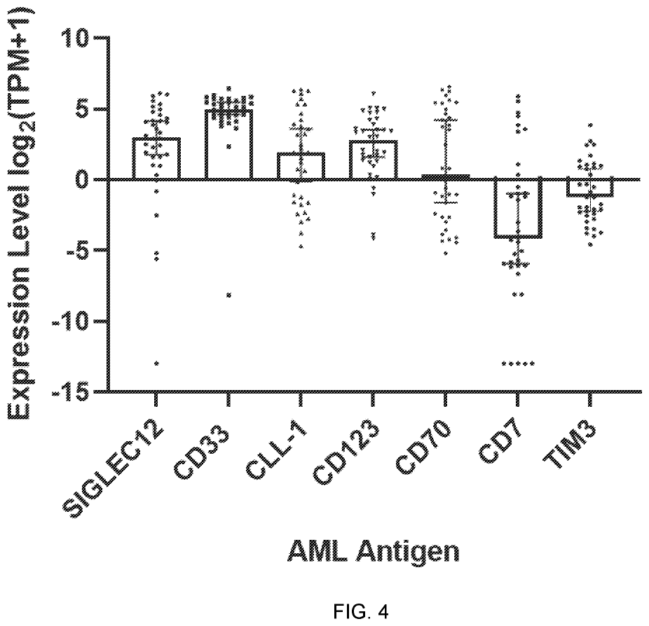

CD33 is known to be highly expressed on AML cell lines and AML blasts in AML patients. The CD33-targeted Gemtuzumab Ozogamicin (GO, MYLOTARG®, Pfizer, New York, NY) is an antibody drug conjugate (ADC) that uses a calicheamicin warhead. GO is the only currently listed form of immunotherapy for AML. There are several other AML targets known to be expressed on AML cell lines and blasts in patients that are currently being investigated in clinical and preclinical studies as potential immunotherapeutic targets. Further RNAseq database analysis demonstrated that siglec12 expression levels in AML cell lines compared quite favorably to mRNA levels of other AML targets [based on 2020 Broad Institute CCLE (Cancer Cell Line Encyclopedia) mRNA expression (RNAseq) data, see FIG. 4]. CD33 expression levels on AML cell lines were comparable to those of siglec12, supporting designation of siglec12 as a possible target for immunotherapeutic development and treatment of AML.

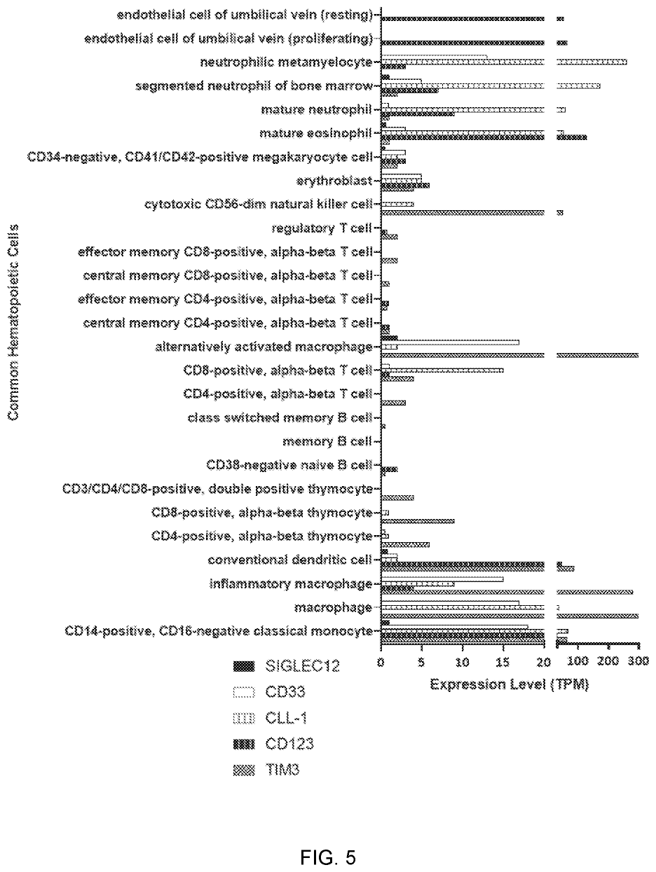

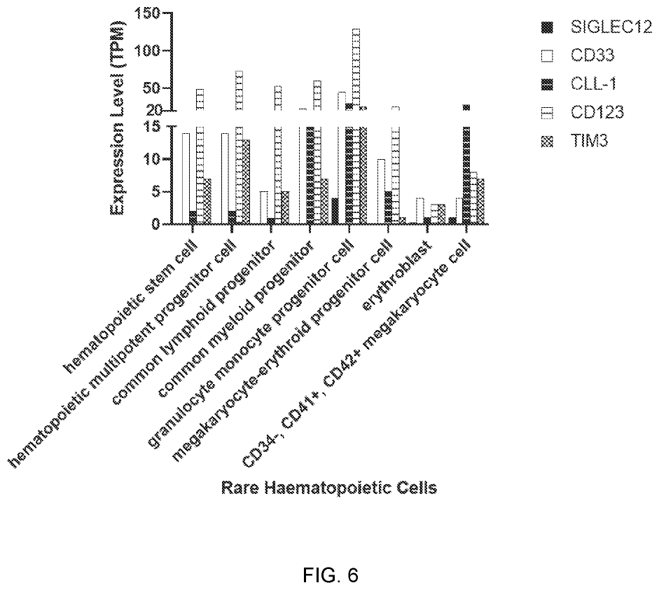

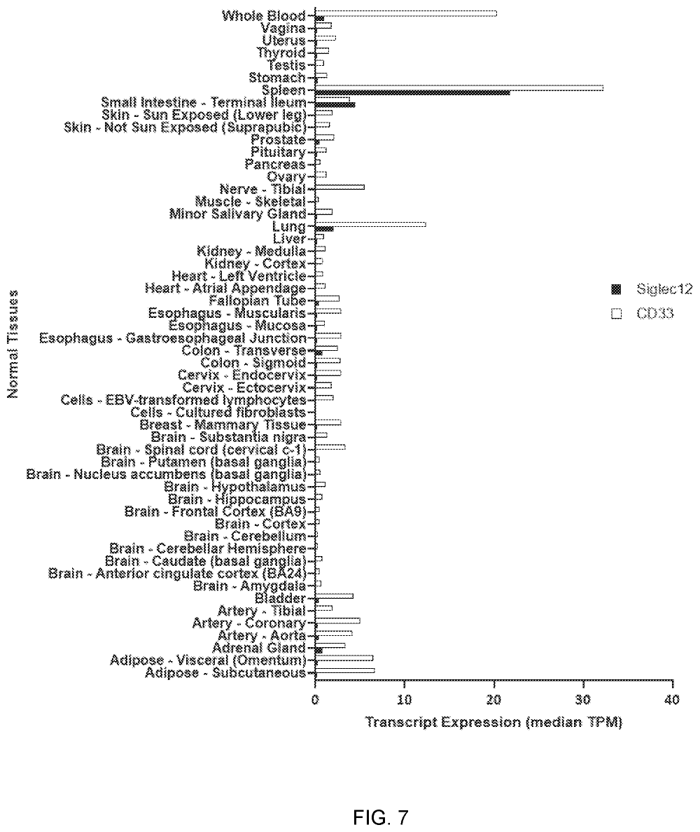

While AML target over expression on AML blasts is important when evaluating potential immunotherapeutic targets, expression on normal tissues and hematopoietic cells can lead to toxicity and dose-limiting requirements. GO has on-target hematological toxicities associated with CD33 expression on hematopoietic progenitor cells that manifests as persistent thrombocytopenia and prolonged neutropenia (Castaigne, S. et al. Lancet. 2012, 379(9825):1508-16). FIG. 5 shows mRNA expression levels of siglec12 and other AML target proteins in common hematopoietic cells based on analysis of public mRNA expression data (Blueprint Consortium). Siglec12 is mainly expressed, albeit at relatively low levels, in classical monocytes, macrophage and alternatively activated macrophage. FIG. 6 shows expression levels of siglec12 and other AML target proteins in hematopoietic progenitor and stem cells based on analysis of public mRNA expression data (Blueprint Consortium). These cell types are rare, but critical with respect to common hematopoietic toxicities. A significant difference was observed between siglec12 and other AML targets with siglec12 having exceptionally low, or no detectable expression in these hematopoietic stem and progenitor cells. The combination of high over expression of siglec12 on AML cells and lack of expression on hematopoietic stem and progenitor cells supports designation of siglec12 as an exemplary target for AML immunotherapeutic development. Siglec12 and CD33 mRNA expression was compared in normal tissues based on analysis of public data (The Genotype-Tissue Expression Project) and again demonstrated relatively low expression of siglec12 in normal tissue (see FIG. 7), supporting an expected advantage of lower on-target toxicities associated with systemic exposure to siglec12-targeting immunotherapeutics. Overall, expression profile results from public RNAseq database analysis indicate that siglec12 is an ideal target for antibody-based immunotherapeutic development and use for the treatment of siglec12-expressing AML, CML, and solid tumors.

### Example 2. Preparation of Antibodies Targeting Siglec12

Antibodies specific for siglec12 were generated by immunizing Trianni transgenic mice (United States Publication Number US2013/0219535). Endogenous immunoglobulin VH, DH and JH; Vκ and Jκ; and Vλ and Jλ gene segments have been replaced in these mice by the full repertoire of human counterpart gene segments. The human gene coding sequences are flanked by mouse cis-acting regulatory elements and recombination signal sequences allowing for a “normal” immune response, including isotype switching with heavy and light chain affinity maturation. The resulting human-mouse chimera antibodies contain human antibody variable domains flanked by mouse heavy and light chain immunoglobulin constant regions. These antibodies are more readily converted to fully human antibodies using standard methods to combine the human variable domains with human constant regions.

Trianni animals were immunized with an antigen prepared by fusion of soluble human siglec12 extracellular (ecto) domain (S12ecto, UniProtKB Q96PQ1, amino acids 1-481, SEQ ID NO: 171) with a mouse Fc2a fragment (SEQ ID NO: 177), the resulting fusion protein referred to herein as “S12-Fc.” Mouse Fc was used in place of commonly used human Fc to minimize generation of antibodies against the Fc region. For S12-Fc preparation, DNA encoding full length human S12ecto was codon optimized, synthesized, and inserted into plasmid vectors for transient transfection and expression in mammalian cells. Expressed fusion protein was purified using protein A/G SEPHAROSE®.

After 6 weeks of immunization, serum samples from immunized mice were screened for the presence of siglec12-specific immunoglobulins. Screening was carried out by enzyme-linked immunosorbent assay (ELISA) and by fluorescence-associated cell sorting (FACS). ELISA assays utilized assay plates coated with S12-Fc target antigen for immobilization of siglec12-specific immunoglobulins from mouse serum samples, which were detected using anti-mouse light chain (kappa and lambda) secondary antibody conjugated to horseradish peroxidase (HRP). The level of bound secondary antibody was assessed by incubation with colorimetric peroxidase substrate followed by measurement of light absorbance intensity values obtained at 450 nm. For FACS analysis, serum samples were combined with U937 cells (Fc receptor blocked with 10% human serum), an AML cell line that overexpresses human siglec12. Immunoglobulin binding to siglec12 associated with these cells is a strong indicator of potential immunotherapeutic use to treat AML. Anti-mouse heavy chain fluorescent detection reagents were used to label cell surface-bound serum immunoglobulins before FACS analysis. Lymph node and spleen cells from the three best responders were harvested and fused with P3X63Ag8.653 mouse myeloma cells. After 10-14 days of selection, the resulting hybridoma library was single-cell cloned into 96-well culture plates using FACS. After 10-14 days of single cell expansion, all monoclonal hybridoma culture supernatants were screened for S12-Fc binding immunoglobulins by ELISA. Supernatant positive for S12-Fc-binding immunoglobulins was further screened by FACS for immunoglobulin binding to U937 cells. Results are shown in Table 12. The Table includes an antibody identification number (ID #) assigned to siglec12-binding antibodies associated with each hybridoma culture. For ELISA results, background adjusted mean absorbance values obtained at 450 nm are shown. For FACS analysis, Flow Fluor MFI is the mean fluorescence intensity from flow cytometry assay (background MFI=100) and flow % gated is the percentage of cells having greater than background fluorescence.

TABLE 12
Antibody screening analysis

| | S12-Fc | Flow | Flow % |
|---|---|---|---|
| ID# | ELISA | Fluor MFI | Gated |
| S0001 | 3.47 | 111 | 8 |
| S0002 | 0.78 | 101 | 5 |
| S0003 | 0.53 | 100 | 4 |
| S0004 | 0.54 | 103 | 5 |
| S0005 | 0.53 | 102 | 4 |
| S0006 | 0.56 | 103 | 3 |
| S0007 | 0.91 | 95 | 2 |
| S0008 | 0.52 | 103 | 5 |
| S0009 | 0.55 | 102 | 5 |
| S0010 | 3.09 | 103 | 3 |
| S0011 | 3.13 | 100 | 4 |
| S0012 | 3.01 | 102 | 4 |
| S0013 | 0.50 | 101 | 5 |
| S0014 | 1.03 | 96 | 3 |
| S0015 | 0.61 | 98 | 3 |
| S0016 | 3.04 | 136 | 18 |
| S0017 | 2.98 | 123 | 11 |
| S0018 | 3.08 | 102 | 3 |
| S0019 | 0.54 | 101 | 4 |
| S0020 | 2.35 | 102 | 4 |
| S0021 | 0.62 | 101 | 4 |
| S0022 | 0.57 | 104 | 6 |
| S0023 | 1.66 | 93 | 3 |
| S0024 | 0.53 | 104 | 4 |
| S0025 | 3.31 | 134 | 17 |
| S0026 | 0.50 | 101 | 5 |
| S0027 | 0.58 | 100 | 3 |
| S0028 | 0.59 | 103 | 6 |
| S0029 | 0.57 | 97 | 4 |
| S0030 | 0.62 | 105 | 6 |
| S0031 | 0.54 | 105 | 6 |
| S0032 | 0.70 | 104 | 5 |
| S0033 | 3.19 | 157 | 28 |
| S0034 | 0.57 | 108 | 7 |
| S0035 | 0.50 | 102 | 4 |
| S0036 | 0.56 | 103 | 5 |
| S0037 | 0.52 | 104 | 5 |
| S0038 | 0.57 | 108 | 7 |
| S0039 | 3.03 | 108 | 6 |
| S0040 | 1.95 | 111 | 6 |
| S0041 | 2.03 | 157 | 27 |
| S0042 | 1.99 | 107 | 4 |
| S0043 | 0.59 | 106 | 6 |
| S0044 | 3.31 | 129 | 14 |
| S0045 | 3.25 | 205 | 49 |
| S0046 | 3.18 | 105 | 3 |
| S0047 | 1.59 | 100 | 3 |
| S0048 | 3.10 | 146 | 23 |
| S0049 | 2.23 | 149 | 27 |
| S0050 | 0.60 | 103 | 5 |
| S0051 | 0.61 | 98 | 5 |
| S0052 | 0.61 | 105 | 5 |
| S0053 | 0.61 | 104 | 6 |
| S0054 | 0.72 | 103 | 5 |
| S0055 | 0.68 | 104 | 6 |
| S0056 | 0.52 | 101 | 3 |
| S0057 | 2.64 | 368 | 79 |
| S0058 | 2.91 | 113 | 6 |
| S0059 | 2.03 | 95 | 3 |
| S0060 | 2.99 | 97 | 3 |
| S0061 | 3.05 | 203 | 46 |
| S0062 | 1.46 | 100 | 2 |
| S0063 | 1.91 | 97 | 3 |
| S0064 | 0.65 | 106 | 7 |
| S0065 | 1.69 | 108 | 7 |
| S0066 | 2.75 | 101 | 4 |
| S0067 | 3.27 | 127 | 13 |
| S0068 | 2.85 | 103 | 4 |
| S0069 | 0.50 | 103 | 5 |
| S0070 | 3.20 | 144 | 20 |
| S0071 | 2.71 | 104 | 5 |
| S0072 | 1.83 | 104 | 5 |
| S0073 | 0.55 | 106 | 6 |
| S0074 | 2.72 | 134 | 17 |
| S0075 | 1.20 | 104 | 4 |
| S0076 | 1.60 | 101 | 5 |
| S0077 | 1.32 | 99 | 4 |
| S0078 | 0.50 | 102 | 6 |
| S0079 | 3.16 | 171 | 37 |
| S0080 | 0.56 | 111 | 8 |
| S0081 | 0.54 | 102 | 5 |
| S0082 | 2.88 | 219 | 54 |
| S0083 | 3.01 | 151 | 25 |
| S0084 | 3.29 | 151 | 25 |
| S0085 | 1.61 | 105 | 4 |
| S0086 | 2.90 | 156 | 28 |
| S0087 | 3.02 | 128 | 12 |
| S0088 | 3.00 | 246 | 63 |
| S0089 | 2.90 | 104 | 3 |
| S0090 | 0.51 | 105 | 6 |
| S0091 | 0.70 | 103 | 5 |
| S0092 | 3.28 | 164 | 34 |
| S0093 | 3.15 | 100 | 4 |
| S0094 | 3.33 | 161 | 27 |
| S0095 | 0.76 | 103 | 5 |
| S0096 | 0.58 | 106 | 4 |
| S0097 | 3.26 | 138 | 17 |
| S0098 | 0.68 | 141 | 21 |
| S0099 | 1.75 | 99 | 3 |
| S0100 | 0.52 | 103 | 5 |
| S0101 | 0.50 | 104 | 3 |
| S0102 | 3.34 | 100 | 3 |
| S0103 | 3.28 | 97 | 2 |
| S0104 | 0.63 | 99 | 3 |
| S0105 | 3.10 | 217 | 49 |
| S0106 | 3.20 | 162 | 28 |
| S0107 | 0.80 | 104 | 5 |
| S0108 | 2.69 | 96 | 2 |
| S0109 | 0.54 | 109 | 7 |
| S0110 | 0.61 | 103 | 4 |
| S0111 | 0.51 | 102 | 4 |
| S0112 | 0.61 | 101 | 3 |
| S0113 | 0.50 | 100 | 3 |

Many of the antibodies tested demonstrated strong association with U937 cell surfaces (flow % gated>10), including S0016, S0017, S0025, S0033, S0041, S0044, S0045, S0048, S0049, S0057, S0061, S0067, S0070, S0074, S0079, S0082, S0083, S0084, S0086, S0087, S0088, S0092, S0094, S0097, S0098, S0105, and S0106.

Antibodies were isolated from mouse ascites fluid or from hybridoma culture media by protein A capture and used for further analysis according to the following examples.

### Example 3. Antibody Characterization by Siglec12 Domain Specificity

ELISA assays were conducted to assess siglec12 domain specificity of antibodies identified during hybridoma screening described above. Antibodies binding to specific subdomains of S12ecto domain could provide certain advantages. For example, it is known that siglec12 expresses two different membrane-bound isoforms: a long form (595 aa, UniProtKB Q96PQ1-1, SEQ ID NO: 172) containing four extracellular domains plus a transmembrane and cytosoplasmic domain; and a shorter isoform (477 aa, UniProtKB Q96PQ1-2, SEQ ID NO: 173) that contains only a single extracellular domain (Vset domain) plus the same transmembrane and cytoplasmic domain.

Antibodies that bind to the Vset2-C2set1 region of siglec12 (S12-V2C1) have the advantage of recognizing both long and short siglec12 isoforms. From a therapeutic standpoint, where both long and short siglec12 isoforms are expressed on a tumor cell, a S12-V2C1-binding antibody will bind a greater number of siglec12 molecules on the cell surface of a targeted cell, thus increasing the sensitivity and efficacy of the therapy.

Antibodies that specifically bind to the Vset1 region of siglec12 (S12-V1) have the advantage of discriminating between the long and short siglec12 isoforms. Such antibodies may be useful for targeting tumor cells expressing only the long isoform of siglec12, while not targeting normal cells expressing the short form of siglec12. S12-V1 antibodies may also be useful from a biomarker standpoint for discriminating between long and short siglec12 isoforms.

Antibodies that specifically bind to the C2 region of siglec12 (S12-C2) may be advantageous in targeting a region of the siglec12 extracellular domain that is closer to the cell surface. Binding to cancer epitopes closer to the cell surface has been shown to provide greater sensitivity with respect to cell cytotoxicity in the context of a CD3-mediated bispecific antibody (e.g., see Estey E.H. et al., Am J Hematol. 2018. 93:1267-91), thus S12-C2-specific antibodies may be candidates for S12-CD3 bispecific antibody development and immunotherapy.

Proteins corresponding with subdomains of S12ecto domain were prepared for use as ELISA target antigens. The subdomains prepared included S12-V1 (amino acids 1-142 of UniProtKB Q96PQ1, SEQ ID NO: 174), S12-V2C1 (native leader sequence with amino acids 143-358 of UniProtKB Q96PQ1, SEQ ID NO: 175), and S12-C2 (native leader sequence with amino acids 365-481 of UniProtKB Q96PQ1, SEQ ID NO: 176) domains. The two domains Vset2 and C2set1 were combined since there is a naturally occurring inter-disulfide bond between the Vset2 and C2set1 domains of siglec12. To produce subdomain proteins, plasmids encoding each of S12-V1, S12-V2C1, and S12-C2 were prepared with additional nucleotides encoding a 6-His affinity tag (SEQ ID NO: 179), in addition to a StrepII affinity tag (aa sequence WSHPQFEK (SEQ ID NO: 180)) at subdomain C-termini for purification. Plasmids were transfected into human cell cultures for protein expression and subdomain proteins were isolated from culture media using Ni-IMAC (immobilized metal affinity chromatography) and STREP-TACTIN® SEPHAROSE® (GE Healthcare, Waukesha, WI) affinity chromatography purification.

For ELISA assays, purified subdomain proteins were passively immobilized on assay plates and incubated with media from hybridoma cultures described above before detection of bound anti-subdomain antibodies using anti-mouse (heavy/light) secondary antibody conjugated to horseradish peroxidase (HRP). Levels of bound secondary antibody were assessed by applying colorimetric substrate and measuring absorbance values at 450 nm. Mean absorbance values obtained for antibodies associated with each hybridoma culture are presented in Table 13. Absorbance values greater than 0.07 indicate specific binding.

TABLE 13
Antibody characterization analysis

| ID# | S12-V1 | S12-V2C1 | S12-C2 |
|---|---|---|---|
| S0001 | 3.99 | 0.14 | 0.07 |
| S0002 | 0.05 | 0.05 | 0.06 |
| S0003 | 0.05 | 0.05 | 0.05 |
| S0004 | 0.06 | 0.06 | 0.06 |
| S0005 | 0.05 | 0.05 | 0.05 |
| S0006 | 0.05 | 0.05 | 0.05 |
| S0007 | 0.05 | 0.05 | 0.05 |
| S0008 | 0.05 | 0.05 | 0.05 |
| S0009 | 0.06 | 0.05 | 0.06 |
| S0010 | 0.05 | 0.05 | 0.09 |
| S0011 | 0.05 | 0.07 | 0.13 |
| S0012 | 0.05 | 0.05 | 0.13 |
| S0013 | 0.05 | 0.05 | 0.05 |
| S0014 | 0.05 | 0.05 | 0.05 |
| S0015 | 0.05 | 0.05 | 0.05 |
| S0016 | 0.05 | 0.61 | 0.05 |
| S0017 | 0.05 | 0.59 | 0.05 |
| S0018 | 0.05 | 0.05 | 0.13 |
| S0019 | 0.06 | 0.06 | 0.06 |
| S0020 | 0.05 | 0.05 | 0.09 |
| S0021 | 0.05 | 0.05 | 0.06 |
| S0022 | 0.05 | 0.05 | 0.06 |
| S0023 | 0.05 | 0.15 | 0.05 |
| S0024 | 0.06 | 0.06 | 0.07 |
| S0025 | 0.05 | 0.57 | 0.05 |
| S0026 | 0.05 | 0.05 | 0.06 |
| S0027 | 0.05 | 0.05 | 0.05 |
| S0028 | 0.05 | 0.05 | 0.05 |
| S0029 | 0.06 | 0.06 | 0.06 |
| S0030 | 0.05 | 0.05 | 0.06 |
| S0031 | 0.05 | 0.05 | 0.06 |
| S0032 | 0.05 | 0.06 | 0.05 |
| S0033 | 0.05 | 0.69 | 0.05 |
| S0034 | 0.06 | 0.05 | 0.06 |
| S0035 | 0.05 | 0.05 | 0.05 |
| S0036 | 0.06 | 0.06 | 0.07 |
| S0037 | 0.05 | 0.06 | 0.05 |
| S0038 | 0.05 | 0.05 | 0.05 |
| S0039 | 3.52 | 0.10 | 0.06 |
| S0040 | 0.05 | 0.05 | 0.12 |
| S0041 | 0.05 | 0.56 | 0.05 |
| S0042 | 0.10 | 0.05 | 0.05 |
| S0043 | 0.06 | 0.06 | 0.06 |
| S0044 | 0.05 | 0.58 | 0.05 |
| S0045 | 0.05 | 0.64 | 0.05 |
| S0046 | 0.05 | 0.05 | 0.12 |
| S0047 | 0.05 | 0.14 | 0.05 |
| S0048 | 0.05 | 0.62 | 0.06 |
| S0049 | 0.05 | 0.55 | 0.05 |
| S0050 | 0.06 | 0.06 | 0.07 |
| S0051 | 0.05 | 0.05 | 0.05 |
| S0052 | 0.06 | 0.06 | 0.06 |
| S0053 | 0.06 | 0.06 | 0.06 |
| S0054 | 0.06 | 0.06 | 0.06 |
| S0055 | 0.05 | 0.06 | 0.05 |
| S0056 | 0.06 | 0.06 | 0.06 |
| S0057 | 0.05 | 0.62 | 0.05 |
| S0058 | 0.30 | 0.27 | 0.27 |
| S0059 | 0.05 | 0.05 | 0.10 |
| S0060 | 0.06 | 0.05 | 0.11 |
| S0061 | 0.05 | 0.65 | 0.05 |
| S0062 | 0.05 | 0.14 | 0.05 |
| S0063 | 0.05 | 0.14 | 0.05 |
| S0064 | 0.07 | 0.07 | 0.07 |
| S0065 | 0.05 | 0.12 | 0.06 |
| S0066 | 0.05 | 0.05 | 0.13 |
| S0067 | 0.06 | 0.57 | 0.05 |
| S0068 | 0.05 | 0.05 | 0.11 |
| S0069 | 0.06 | 0.06 | 0.06 |
| S0070 | 0.05 | 0.56 | 0.05 |
| S0071 | 0.05 | 0.05 | 0.11 |
| S0072 | 0.19 | 0.18 | 0.21 |
| S0073 | 0.07 | 0.08 | 0.07 |
| S0074 | 0.05 | 0.63 | 0.05 |
| S0075 | 0.05 | 0.14 | 0.05 |
| S0076 | 0.05 | 0.13 | 0.05 |
| S0077 | 0.05 | 0.13 | 0.05 |
| S0078 | 0.05 | 0.05 | 0.06 |
| S0079 | 0.05 | 0.64 | 0.05 |
| S0080 | 0.06 | 0.05 | 0.05 |
| S0081 | 0.05 | 0.05 | 0.05 |
| S0082 | 0.05 | 0.61 | 0.05 |
| S0083 | 0.05 | 0.64 | 0.05 |
| S0084 | 0.05 | 0.62 | 0.05 |
| S0085 | 0.05 | 0.13 | 0.05 |
| S0086 | 0.05 | 0.56 | 0.05 |
| S0087 | 0.05 | 0.56 | 0.05 |
| S0088 | 0.05 | 0.58 | 0.05 |
| S0089 | 0.12 | 0.05 | 0.06 |
| S0090 | 0.06 | 0.06 | 0.06 |
| S0091 | 0.07 | 0.07 | 0.09 |
| S0092 | 0.05 | 0.56 | 0.05 |
| S0093 | 0.05 | 0.05 | 0.11 |
| S0094 | 0.05 | 0.54 | 0.05 |
| S0095 | 0.06 | 0.06 | 0.06 |
| S0096 | 0.06 | 0.06 | 0.06 |
| S0097 | 0.06 | 0.53 | 0.06 |
| S0098 | 0.11 | 0.11 | 0.13 |
| S0099 | 0.06 | 0.14 | 0.08 |
| S0100 | 0.07 | 0.07 | 0.07 |
| S0101 | 0.08 | 0.08 | 0.07 |
| S0102 | 0.06 | 1.65 | 0.08 |
| S0103 | 0.06 | 0.06 | 0.13 |
| S0104 | 0.07 | 0.07 | 0.07 |
| S0105 | 0.06 | 0.48 | 0.06 |
| S0106 | 0.06 | 0.42 | 0.06 |
| S0107 | 0.06 | 0.06 | 0.07 |
| S0108 | 0.06 | 0.06 | 0.12 |
| S0109 | 0.06 | 0.07 | 0.08 |
| S0110 | 0.08 | 0.08 | 0.10 |
| S0111 | 0.07 | 0.07 | 0.08 |
| S0112 | 0.07 | 0.07 | 0.07 |
| S0113 | 0.07 | 0.07 | 0.08 |
| S0114 | 0.17 | 1.61 | 0.20 |

### Example 4. Antibody Characterization by Inter-Siglec Specificity

ELISA assays were also conducted to assess inter-siglec antibody specificity for other CD33-related siglecs. Siglec12 is a member of the CD33-related siglec family. From the family, human siglec7 and siglec9 have the greatest sequence identity to human siglec12. Specificity for siglec12 over other CD33-related siglecs by candidate antibodies is a desirable property for reducing potential toxicity associated with off-target binding.

Fusion proteins of human siglec7 or siglec9 ectodomains fused with human Fc domains (R&D Systems, Minneapolis, MN) were immobilized on assay plates and incubated with media from hybridoma cultures described above before detection of bound antibodies using anti-mouse (heavy/light) secondary antibody conjugated to HRP. Levels of bound secondary antibody were assessed by applying colorimetric substrate and measuring absorbance values at 450 nm. Mean absorbance values obtained for antibodies associated with each hybridoma culture are presented in Table 14.

TABLE 14
Antibody inter-siglec specificity analysis

| ID# | S7ecto-Fc | S9ecto-Fc |
|---|---|---|
| S0001 | 3.52 | 0.07 |
| S0002 | 0.06 | 0.06 |
| S0003 | 0.06 | 0.06 |
| S0004 | 0.07 | 0.06 |
| S0005 | 0.06 | 0.06 |
| S0006 | 0.06 | 0.05 |
| S0007 | 0.05 | 0.05 |
| S0008 | 0.08 | 0.05 |
| S0009 | 0.06 | 0.06 |
| S0010 | 0.05 | 0.05 |
| S0011 | 0.05 | 0.05 |
| S0012 | 0.05 | 0.05 |
| S0013 | 0.06 | 0.06 |
| S0014 | 0.05 | 0.05 |
| S0015 | 0.05 | 0.05 |
| S0016 | 0.06 | 0.06 |
| S0017 | 0.06 | 0.06 |
| S0018 | 0.05 | 0.05 |
| S0019 | 0.08 | 0.07 |
| S0020 | 0.05 | 0.05 |
| S0021 | 0.07 | 0.06 |
| S0022 | 0.07 | 0.06 |
| S0023 | 0.05 | 0.05 |
| S0024 | 0.07 | 0.06 |
| S0025 | 0.06 | 0.06 |
| S0026 | 0.06 | 0.07 |
| S0027 | 0.07 | 0.07 |
| S0028 | 0.06 | 0.06 |
| S0029 | 0.07 | 0.07 |
| S0030 | 0.06 | 0.07 |
| S0031 | 0.06 | 0.06 |
| S0032 | 0.06 | 0.07 |
| S0033 | 0.07 | 0.08 |
| S0034 | 0.07 | 0.07 |
| S0035 | 0.06 | 0.07 |
| S0036 | 0.07 | 0.08 |
| S0037 | 0.09 | 0.07 |
| S0038 | 0.06 | 0.06 |
| S0039 | 3.55 | 0.06 |
| S0040 | 0.05 | 0.05 |
| S0041 | 0.06 | 0.06 |
| S0042 | 0.06 | 0.05 |
| S0043 | 0.10 | 0.09 |
| S0044 | 0.06 | 0.07 |
| S0045 | 0.08 | 0.07 |
| S0046 | 0.06 | 0.05 |
| S0047 | 0.05 | 0.05 |
| S0048 | 0.08 | 0.08 |
| S0049 | 0.06 | 0.07 |
| S0050 | 0.08 | 0.08 |
| S0051 | 0.05 | 0.05 |
| S0052 | 0.08 | 0.07 |
| S0053 | 0.07 | 0.07 |
| S0054 | 0.07 | 0.07 |
| S0055 | 0.06 | 0.06 |
| S0056 | 0.08 | 0.07 |
| S0057 | 0.07 | 0.08 |
| S0058 | 0.42 | 0.34 |
| S0059 | 0.06 | 0.05 |
| S0060 | 0.06 | 0.05 |
| S0061 | 0.06 | 0.07 |
| S0062 | 0.05 | 0.05 |
| S0063 | 0.05 | 0.05 |
| S0064 | 0.09 | 0.08 |
| S0065 | 0.06 | 0.05 |
| S0066 | 0.06 | 0.05 |
| S0067 | 0.06 | 0.07 |
| S0068 | 0.06 | 0.05 |
| S0069 | 0.07 | 0.07 |
| S0070 | 0.07 | 0.08 |
| S0071 | 0.06 | 0.05 |
| S0072 | 0.25 | 0.23 |
| S0073 | 0.08 | 0.09 |
| S0074 | 0.06 | 0.07 |
| S0075 | 0.05 | 0.05 |
| S0076 | 0.05 | 0.05 |
| S0077 | 0.05 | 0.05 |
| S0078 | 0.06 | 0.07 |
| S0079 | 0.07 | 0.09 |
| S0080 | 0.06 | 0.07 |
| S0081 | 0.06 | 0.06 |
| S0082 | 0.06 | 0.07 |
| S0083 | 0.07 | 0.09 |
| S0084 | 0.08 | 0.11 |
| S0085 | 0.05 | 0.05 |
| S0086 | 0.07 | 0.06 |
| S0087 | 0.07 | 0.07 |
| S0088 | 0.06 | 0.08 |
| S0089 | 0.07 | 0.06 |
| S0090 | 0.08 | 0.07 |
| S0091 | 0.12 | 0.11 |
| S0092 | 0.06 | 0.07 |
| S0093 | 0.05 | 0.05 |
| S0094 | 0.07 | 0.06 |
| S0095 | 0.08 | 0.08 |
| S0096 | 0.08 | 0.07 |
| S0097 | 0.08 | 0.07 |
| S0098 | 0.23 | 0.19 |
| S0099 | 0.05 | 0.06 |
| S0100 | 0.08 | 0.07 |
| S0101 | 0.07 | 0.08 |
| S0102 | 0.07 | 0.06 |
| S0103 | 0.06 | 0.05 |
| S0104 | 0.07 | 0.07 |
| S0105 | 0.07 | 0.07 |
| S0106 | 0.08 | 0.08 |
| S0107 | 0.07 | 0.06 |
| S0108 | 0.05 | 0.05 |
| S0109 | 0.06 | 0.06 |
| S0110 | 0.08 | 0.08 |
| S0111 | 0.07 | 0.07 |
| S0112 | 0.07 | 0.07 |
| S0113 | 0.08 | 0.08 |

Of the antibodies with strong association with U937 cell surfaces (flow % gated>10), most demonstrated lack of specificity for siglec7 and siglec9 (A450>0.09), including S0016, S0017, S0025, S0033, S0041, S0044, S0045, S0048, S0049, S0057, S0061, S0067, S0070, S0074, S0079, S0082, S0083, S0086, S0087, S0088, S0092, S0094, S0097, S0105, and S0106.

### Example 5. Siglec12-Dependent Internalization

Internalization of siglec 12-bound anti-siglec12 antibodies was assessed in U937 AML cells using anti-siglec12 antibodies described in Example 2. U937 AML cells were cultured and duplicate wells were incubated with or without anti-siglec12 antibodies (described in Example 2) or anti-CD33 antibody (P67.6, BioLegend, San Diego, CA) on ice for 1 hr. Cells were washed and resuspended in 800 μL ice cold complete media (10% FBS, RPMI) before plating 100 μL per well in 96-well plates. Cells were then incubated at 37° C. and 5% CO₂ to promote internalization. Internalization was halted at 15, 30, 45, 60, 90, and 120 minute time points by placement on ice before cells were harvested and washed with cold PBSB (PBS+0.1% BSA). Harvested cells were then incubated with phycoerythrin (PE)-conjugated goat anti-mouse antibody (1:400, Bethyl Laboratories, Montgomery, TX) for 30 min on ice. Cells were again washed with PBSB and analyzed by flow cytometry using a GUAVA® EASYCYTE™ Plus flow cytometer (MilliporeSigma, Burlington, MA) to assess percent surface expression and percent internalization of antibody-associated siglec12 and CD33 at the time points evaluated. A comparison of average percent surface expression over time between siglec12 (labeled using S0057 antibody) and CD33 is shown in Table 15.

TABLE 15
Percent surface expression over time

| Time point | Siglec12 % surface | CD33 % surface |
|---|---|---|
| (min) | expression | expression |
| 0 | 100% | 100% |
| 15 | 96% | 79% |
| 30 | 84% | 72% |
| 45 | 73% | 68% |
| 60 | 65% | ND |
| 90 | 47% | 53% |
| 120 | 40% | 48% |

The results demonstrate similar internalization levels over time between the two antibody-associated markers. A comparison of percent siglec12 internalization observed at 2 hours with different anti-siglec12 antibodies is presented in Table 16.

TABLE 16
Percent internalization

| ID# | % Internalization |
|---|---|
| S0023 | 32% |
| S0042 | 66% |
| S0057 | 58% |
| S0102 | 12% |
| S0114 | 67% |

The results show internalization activity with each antibody tested, with highest activity among antibodies S0042, S0057, and S0114.

### Example 6. Siglec12 Intracellular Trafficking

Siglec12-antibody complexes were assessed for intracellular trafficking to acidified cell compartments (e.g., the lysome) using acid sensitive dyes. Trafficking of internalized antibody-target complexes to acidified cellular compartments is desirable for some therapeutic antibody applications, including some utilizing antibody-drug conjugates (ADCs). Such trafficking may facilitate ADC drug release and/or activation. ZENON® labeling reagent (Thermo Fisher Scientific, Waltham, MA) includes an anti-mouse Fab fragment labeled with a pH sensitive dye that increases green fluorescence upon acidification. S0057 anti-siglec12 antibody was pre-incubated with 750 nM ZENON® labeling reagent for 15 min in PBSB. 50 nM P67.6 (anti-CD33) was similarly prepared and used for comparison (as internalized CD33-antibody complexes are known to traffic to lysosomes). Resulting complexes were added to separate wells containing U937 cells and incubated for 0, 4, or 24 hours at 37° C. at 5% CO₂. Green fluorescence was measured at each time point using a GUAVA® EASYCYTE™ Plus flow cytometer. Resulting mean fluorescence intensity (MFI) values are presented in Table 17.

TABLE 17
Acidification analysis results

| | |
|---|---|
| Time point | MFI values |

| (hours) | Siglec12 complexes | CD33 complexes |
|---|---|---|
| 0 | 19.96 | 20.83 |
| 4 | 24.69 | 26.01 |
| 24 | 26.48 | 31.94 |

Acidification was comparable between siglec12-bound and CD33-bound complexes validating the potential of siglec12-specific antibody targeting as an option for ADC therapeutics.

### Example 7. ADC Cytotoxicity on AML Cells

Anti-siglec12 antibodies were assessed for utility in delivering cytotoxic payloads to cells. 1 mg S0057 was used to prepare a 3 mg/mL antibody solution in PBS (pH 7.8)/30% propylene glycol and combined with 40 nmols of N-succinimidyl ester activated MYLOTARG® linker/payload (Levena Biopharma, San Diego, CA). MYLOTARG® has an acid-sensitive linker with a calicheamicin payload. The antibody solution was incubated overnight at room temperature under gentle shaking. Resulting S0057-ADC was purified from unconjugated linker/payload by buffer exchange and size-exclusion chromatography. U937 AML cells were plated in a 96-well plate and incubated in complete media (10% FBS, RPMI+100 μg/mL human IgG) with S0057-ADC titrated from 100 to 0.1 nM. Cells were incubated for 3 days at 37° C. and 5% CO2, after which average percent cell viability was determined using GUAVA® VIACOUNT® reagent (Luminex, Austin, TX) and GUAVA® EASYCYTE™ Plus flow cytometer. Average percent viability values with each antibody concentration tested are presented in Table 18.

TABLE 18
Average percent cell viability

| S0057-ADC | Average % cell |
|---|---|
| concentration (nM) | viability |
| 100 | 9.9 |
| 50 | 14.6 |
| 25 | 37.7 |
| 12.5 | 53.4 |
| 6.25 | 57.1 |
| 3.125 | 62.7 |
| 1.5625 | 75.0 |
| 0.78125 | 81.9 |
| 0.390625 | 87.1 |
| 0.195313 | 90.9 |
| 0.097656 | 92.8 |

S0057-ADC concentrations as low as 6.25 nM were capable of reducing cell viability by nearly half.

### Example 8. Siglec12 Detection in AML Bone Marrow

Bone marrow aspirate from patients with AML or acute promyelocytic leukemia (APL, negative control) was fixed with 10% formalin and used to prepare and mount 5-micron thick sections. Antigen retrieval was performed by incubating sections in antigen retrieval buffer (pH 9) for 40 minutes. Sections were treated with blocking buffer with 10% normal horse serum and incubated with S0102 anti-Siglec12 antibody (1:150). Bound antibodies were detected using peroxidase-conjugated secondary antibodies and development with 3,3′-diaminobenzidine (DAB) chromogen. Sections were counterstained with hematoxylin and eosin and visualized microscopically. Siglec12 staining of AML blasts was observed in AML patient bone marrow, while staining was absent in APL patient tissues.

### Example 9. Siglec12 Specificity Evaluation

To verify anti-siglec12 antibody specificity for native siglec12 (over other siglec family members), cross-reactivity analysis was carried out using flow cytometry. Expi 293 cells were grown in 6 well plates and transfected with 2.5 mg of pcDNA3.1 expression vectors (Thermo Fisher Scientific, Waltham, MA) with inserts encoding siglec6, siglec7, siglec8, siglec9, or siglec12 proteins with C-terminal FLAG tags. The following day, Expi293 enhancers were added. On day 2 (post-transfection) cells were washed in PBSB and aliquoted into plastic V-bottomed plates at a density of 5×10⁵ cells/well. An aliquot was also used for Western blot analysis to confirm siglec expression (using anti-FLAG antibody as described below). Plated cells were incubated with anti-siglec12 antibodies at a concentration of 500 nM or siglec-specific control antibody (all from R&D Systems, Minneapolis, MN; Siglec6-specific clone 767329; Siglec8-specific clone 837535; Siglec9-specific clone 191240) at a concentration of 12.5 μg/mL for 2 hours on ice. Cells were then washed with ice cold PBSB and incubated with PE-goat anti mouse H/L chain secondary antibody (1:400 in PBSB) for 30 minutes on ice. Cells were again washed with ice cold PBSB and analyzed for surface staining based on fluorescence intensity determined by GUAVA® EASYCYTE™ Plus flow cytometer. Resulting MFI values are presented in Table 19.

TABLE 19
Surface staining results

| Construct | S0023 | S0042 | S0057 | S0102 |
|---|---|---|---|---|
| Empty Vector | 2.4 | 5.5 | 6.6 | 4.8 |
| Siglec 6 | 29.3 | 25.7 | 29.8 | 28.3 |
| Siglec 7 | 2.1 | 3.9 | 2.8 | 25.1 |
| Siglec 8 | 2.8 | 5.3 | 6.3 | 7.0 |
| Siglec 9 | 3.0 | 2.3 | 1.3 | 28.0 |
| Siglec 12 | 484.5 | 537.8 | 912.0 | 1011.6 |

All anit-siglec12 antibodies tested bound to siglec12-expressing cells and demonstrated little to no binding of other siglec proteins.

For FLAG-specific Western blot analysis, cells were lysed in radioimmunoprecipitation assay (RIPA) buffer and run on 4-12% NUPAGE™ gel (Thermo Fisher Scientific, Waltham, MA). Proteins were transferred onto PVDF membranes and blocked with 3% BSA in PBS. Anti-FLAG primary antibody (anti-FLAG OTI4C5, Origene Biotechnology, Rockville, MD) was diluted 1:2000 in PBS with 0.05% TWEEN® 20 and 0.3% BSA and used to treat PVDF membranes overnight at 4° C. Membranes were washed and incubated with alkaline phosphatase (AP)-labeled goat anti mouse H/L chain secondary antibody for 30 minutes at room temperature. Membranes were washed again and developed with CDP-STAR® chemiluminescent detection reagent for digital imaging of labeled bands. Results confirmed expression of siglec proteins.

### Example 10. S0057 Epitope Evaluation

An antibody binding competition assay was carried out to confirm S0057 epitope and assess epitope overlap with other anti-siglec12 antibodies. A fusion protein of S12 V2C1 (SEQ ID NO: 175) and mouse Fc region fragment m2cFc (SEQ ID NO: 178) was prepared and used to coat 96-well plates in PBS overnight at 4° C. Plates were then blocked with 3% BSA/PBS for one hour before addition of biotinylated S0057 (15 nM) and a titration of unlabeled antibody (200 nM to 6.2 nM of S0057, S0023, S0102, or S0042). Plates were incubated for two hours and washed before incubation with streptavidin-Alkaline phosphatase conjugate (1:5000 in PBS/TWEEN® 20 with 0.3% BSA) for 30 min. Plates were again washed and incubated with CDP-STAR® chemiluminescence substrate before chemiluminescence analysis using a BioTek CYTATION™ 5 instrument (Winooski, VT). Mean luminescence values obtained are presented in Table 20.

TABLE 20
Mean luminescence values

| | Mean luminescence values | | | | |
|---|---|---|---|---|---|
| Unlabeled | No | | | | |
| antibody | competing | Competition | Competition | Competition | Competition |
| concentration | antibody | with | with | with | with |
| (nM) | (control) | S0057 | S0023 | S0102 | S0042 |
| 200 | 2170 | 192 | 2418 | 1815 | 2032 |
| 100 | 2223 | 408 | 2312 | 2045 | 2200 |
| 50 | 2337 | 747 | 2325 | 2104 | 2302 |
| 25 | 2263 | 1077 | 2426 | 2231 | 2383 |
| 12.5 | 2540 | 1401 | 2532 | 2221 | 2190 |
| 6.25 | 2760 | 1930 | 2670 | 2427 | 2264 |
| 0 | 2758 | 2702 | 2777 | 2580 | 2631 |

V2C1-specific antibodies S0023 and S0102 (see Example 3) did not effectively compete with S0057, thus indicating that the S0057 epitope most likely does non-overlap with S0023 or S0102 epitopes. The unlabeled S0057 demonstrated effective competition as a positive control and S0042 (V1-binding negative control) did not compete for binding.

### Example 11. S0057 Binding Affinity

Siglec12 binding kinetics were assessed for S0057 whole antibody and corresponding Fab fragment (obtained by whole antibody digestion). A fusion protein of S12 V2C1 (SEQ ID NO: 175) and mouse Fc region fragment m2cFc (SEQ ID NO: 178) was prepared and used to coat 96-well plates in PBS overnight at 4° C. Plates were then blocked with 3% BSA/PBS for one hour before addition of S0057 Fab fragment or S0057 whole antibody at titrations from 3 μM to 50 pM (in 0.3% BSA/PBS) using 3-fold dilutions. Plates were then incubated for 2.5 hours at room temperature under gentle shaking. Plates were washed and incubated with secondary antibody [1:10,000 goat anti-mouse Kappa chain HRP conjugate (Bethyl Laboratories, Montgomery, TX) in 0.3% BSA/PBS/Triton X-100] for 30 min at room temperature. Plates were again washed before addition of colorimetric peroxidase substrate, quenching, and absorbance measurement at 450 nm. Absorbance values were used to determine equilibrium dissociation constant (Kd) values for Fab and whole antibody formats. S0057 Fab fragment Kd was determined to be 4.8 nM and whole antibody Kd to be 2.8 nM.

While this invention has been particularly shown and described with references to preferred embodiments thereof, it will be understood by those skilled in the art that various changes in form and details may be made therein without departing from the scope of the invention encompassed by the appended claims.

1. An antibody, wherein the antibody binds a sialic acid binding-immunoglobulin type lectin (siglec) and wherein the antibody comprises at least one human antibody variable domain.

2. The antibody of claim 1, wherein the siglec comprises siglec12.

3. The antibody of claim 2 comprising at least one complementarity determining region (CDR) amino acid sequence selected from Table 5.

4. The antibody of claim 3 comprising at least one variable domain comprising a CDR amino acid sequence set selected from Table 6.

5. The antibody of claim 4 comprising a pair of variable domains comprising a CDR amino acid sequence set pair selected from Table 7.

6. The antibody of claim 3 comprising at least one antibody framework (FR) amino acid sequence selected from Table 8.

7. The antibody of claim 1 comprising a variable domain amino acid sequence selected from Table 2 or encoded by a nucleic acid sequence selected from Table 3.

8. The antibody of claim 7 comprising a variable domain amino acid sequence pair selected from Table 4.

9. The antibody of claim 1, wherein the antibody binds to the siglec12 extracellular domain.

10. The antibody of claim 9, wherein the antibody binds to the siglec12 long isoform extracellular domain, the siglec12 short isoform extracellular domain, a subdomain of the siglec12 long isoform extracellular domain, and/or a subdomain of the siglec12 short isoform extracellular domain.

11. The antibody of claim 10, wherein the subdomain of the siglec12 long isoform extracellular domain and/or the subdomain of the siglec12 short isoform extracellular domain comprises the Vset1 (S12-V1) region, the Vset2 (S12-V2) region, the C2set1 (S12-C1) region, or the C2set2 (S12-C2) region.

12. The antibody of claim 9, wherein the antibody binds to a combination of subdomains of the siglec12 extracellular domain.

13. The antibody of claim 12, wherein the combination of subdomains comprises S12-V2 and S12-C1 (S12-V2C1) or S12-V2, S12-C1, and S12-C2 (S12-V2C1C2).

14. The antibody of claim 13, wherein the antibody does not bind to S12-V1 or S12-C2.

15. The antibody of claim 14, wherein the antibody does not bind to siglec7 or siglec9.

16. (canceled)

17. The antibody of claim 1, wherein the siglec is associated with a cancer cell.

18. (canceled)

19. The antibody of claim 17, where the cancer cell is from a cancer selected from any of those listed in Table 10.

20. The antibody of claim 17, wherein the cancer cell is a leukemia cell, and wherein the leukemia cell comprises an acute myeloid leukemia (AML) cell or a chronic myeloid leukemia (CML) cell.

21-26. (canceled)

27. The antibody of claim 1, wherein the antibody comprises a CD3 binding domain, a chimeric antigen receptor, or a conjugate, or any combination of the above.

28-34. (canceled)

35. A method of treating a therapeutic indication in a subject, the method comprising administering the antibody of claim 1 to a subject in need.

36. The method of claim 35, wherein the therapeutic indication comprises a cancer-related indication.

37-51. (canceled)

52. A method of treating a subject with cancer, the method comprising administering an anti-siglec12 antibody to the subject, the anti-siglec12 antibody comprising:
a heavy chain variable domain (VH) comprising an amino acid sequence selected from the group consisting of SEQ ID NOs: 1-7; and
a light chain variable domain (VL) comprising an amino acid sequence selected from the group consisting of SEQ ID NOs: 8-14.

53-58. (canceled)

59. The method of claim 52, wherein the VH comprises the amino acid sequence of SEQ ID NO: 4 and the VL comprises the amino acid sequence of SEQ ID NO: 11.

60. The method of claim 59, wherein the anti-siglec12 antibody comprises a format selected from the group consisting of a Fab format and a whole antibody format.

61. The method of claim 60, wherein the equilibrium dissociation constant (Kd) for binding of the anti-siglec12 antibody to siglec12 is from about 1 nM to about 10 nM.
